SEQUENCE LISTING (23 sequences)

SEQ ID NO: 1 - protein, 12 aa, synthetic construct
HHLGGAKQAGDV

SEQ ID NO: 2 - protein, 5 aa, synthetic construct
CREKA

SEQ ID NO: 3 - protein, 11 aa, synthetic construct
  MOD_RES 11: note = amidated C terminus
GPRPPGGSKGC

SEQ ID NO: 4 - protein, 12 aa, synthetic construct
GLPCDYYGTCLD

SEQ ID NO: 5 - protein, 12 aa, synthetic construct
GYLCGDYTLCPD

SEQ ID NO: 6 - protein, 13 aa, synthetic construct
  MOD_RES 1: note = acetylated N terminus
  SITE 2: note = D-glutamic acid
  MOD_RES 4: note = hydroxyproline
  MOD_RES 13: note = amidated C terminus
YECPYGLCYIQGK

SEQ ID NO: 7 - protein, 6 aa, synthetic construct
  MOD_RES 5: note = 9-fluorenylmethyloxycarbonyl lysine
KCREKA

SEQ ID NO: 8 - protein, 13 aa, synthetic construct
  MOD_RES 1: note = acetylated N terminus
GNQEQVSPLTLLK

SEQ ID NO: 9 - protein, 12 aa, synthetic construct
WFHCPYDLCHIL

SEQ ID NO: 10 - protein, 12 aa, synthetic construct
AFHCPYDLCHIL

SEQ ID NO: 11 - protein, 12 aa, synthetic construct
WAHCPYDLCHIL

SEQ ID NO: 12 - protein, 12 aa, synthetic construct
WFACPYDLCHIL

SEQ ID NO: 13 - protein, 12 aa, synthetic construct
WFHCAYDLCHIL

SEQ ID NO: 14 - protein, 12 aa, synthetic construct
WFHCPADLCHIL

SEQ ID NO: 15 - protein, 12 aa, synthetic construct
WFHCPYALCHIL

SEQ ID NO: 16 - protein, 12 aa, synthetic construct
WFHCPYDACHIL

SEQ ID NO: 17 - protein, 12 aa, synthetic construct
WFHCPYDLCAIL

SEQ ID NO: 18 - protein, 12 aa, synthetic construct
WFHCPYDLCHAL

SEQ ID NO: 19 - protein, 12 aa, synthetic construct
WFHCPYDLCHIA

SEQ ID NO: 20 - protein, 12 aa, synthetic construct
QWECPYGLCWIQ

SEQ ID NO: 21 - protein, 12 aa, synthetic construct
QAECPYGLCWIQ

SEQ ID NO: 22 - protein, 12 aa, synthetic construct
QWECPYGLCAIQ

SEQ ID NO: 23 - protein, 211 aa, synthetic construct [sequence omitted]

(19) United States
(12) Patent Application Publication
(10) Pub. No.: US 2026/0048022 A1
(43) Pub. Date: Feb. 19, 2026

(54) ENDOTHELIAL DAMAGE AND NANOPARTICLE TARGETING: COMPOSITIONS, PROCESSES, USES

(71) Applicant: Nanoprobes, Inc., Yaphank, NY (US)

(72) Inventors: James Frederick HAINFELD, Shoreham, NY (US); Rose Phorogh RAZAVI, Manhasset, NY (US); Michael J. O'CONNOR, East Setauket, NY (US)

(21) Appl. No.: 18/865,969

(22) PCT Filed: May 12, 2023
(86) PCT No.: PCT/US2023/066963

§ 371 Date: Nov. 14, 2024

Related U.S. Application Data
(60) Provisional application No. 63/342,119, filed on May 15, 2022.

(51) Int. Cl. A61K 9/51; A61K 9/00; A61K 31/337; A61K 31/4745; A61P 35/00

(52) U.S. Cl. CPC A61K 9/5169; A61K 9/0019; A61K 9/5192; A61K 31/337; A61K 31/4745; A61P 35/00

(57) ABSTRACT

A novel targeting process and nanoparticle design is disclosed, improving delivery of drugs and other pay load materials to tumors and other sites of interest. In a preferred embodiment, endothelial cells are damaged at the target site, thereby activating platelets. Nanoparticles bearing fibrinogen or other materials that bind to activated platelets are administered that also contain one or more drugs or other pay load substances thereby improving payload delivery to the targeted site.

### CROSS REFERENCE TO RELATED APPLICATIONS

This application claims the priority benefit of U.S. Provisional Application No. 63/342,119 filed on May 15, 2022, the contents of which are incorporated herein by reference in its entirety.

### REFERENCE TO AN ELECTRONIC SEQUENCE LISTING

The contents of the electronic sequence listing (283442000140seqlist.xml; Size: 23,825 bytes; and Date of Creation: May 2, 2023) are herein incorporated by reference in their entirety.

### FIELD OF THE INVENTION

The present invention relates to compositions comprising nanoparticles and methods of treating diseases and disorders using targeted drug delivery.

### BACKGROUND OF THE INVENTION

Many drugs and materials with desirable effects have been developed for specific cells, tissues, or organs. However, their full desired effects cannot often be achieved due to off-target accumulation, creating toxicity or side effects which limits the dose to the desired target. In addition to cancer, many other ailments could benefit from more a targeted drug delivery to specific areas of interest, such as coronary artery disease, stroke, respiratory infections, chronic obstructive pulmonary disease (COPD), diabetes, diarrheal diseases, tuberculosis, cirrhosis, arthritis, obesity, spinal cord damage, hemorrhage, tissue damage, arthritis, Parkinson's disease, and Alzheimer's. For example, medications that treat plaque buildup in the circulatory system could be better targeted, potentially reducing deaths from heart attacks.

Therefore, it is of intense interest to better target drugs or other materials to the intended tissue. However, thus far, only a small percentage of intravenously injected drugs or materials reach their intended target. For example, Dubey et al. linked the drug Gemcitabine to hyaluronic acid to target tumors expressing CD44 (a natural hyaluronic acid receptor ligand), but found only 1.5% ID/g (injected dose per gram tissue) localized at the tumors 24 hours after treatment administration. Hence, only 1.5% of the injected dose was delivered for a 1 g tumor. Yet, the rate of tumor growth decreased despite low drug delivery. In another study by Jin et al. 2013, a luminescent nanoparticle for tumor imaging was targeted to tumors using an RGD peptide which binds to many tumors. By adding RGD targeting, nanoparticle localization to tumor increased from 3.0% ID/g of administered dose to 14.5% ID/g. Zhong et al., 2015, used folic acid (since many tumors have upregulated folic acid receptors) attached to gold nanorods, and found 17% ID/g tumor accumulation after 24 hrs. In another example, radioisotopes were attached to tumor specific antibodies, but the maximal tumor uptake was found to be 12-14% ID/g (Grunberg et al., 2005). Adams et al., 2001, found that high affinity scFv antibody fragments had only 1.4% ID/g accumulation on tumors. Rossin et al., 2018, linked the drug monomethyl auristatin E, (a tubulin-binding antimitotic) to a monoclonal antibody (CC49) that targets tumor-associated glycoprotein-72 (TAG72). TAG72 expression in normal adult tissues is very low, but it is widely expressed in a range of epithelial-derived human adenocarcinomas, including breast, colorectal, stomach, lung, pancreatic, prostate, and ovarian cancers. Despite high TAG72 expression in tumors, only 6% ID/g was found targeted to ovarian tumor zenografts. Banergee et al., 2016, targeted lipid particles containing paclitaxel to orthotopic brain tumors in rats using Tyr-3-octreotide (TOC), which binds somatostatin receptors (SSTRs) that are over-expressed in gliomas. Yet only 0.65% ID/g of administered dose was found at the tumor.

In some cases, active targeting decreased tumor uptake. For example, Chauhan et al., 2018, found that a photothermal gold particles without active targeting had tumor uptake of 2.3% ID/g (due to tumor leakiness), but when targeted with folic acid (an active targeting molecule), tumor uptake decreased to 0.3% ID/g. Kim et al. 2015, used Low-Density Lipoprotein-Mimetic Solid Lipid Nanoparticles carrying the drug paclitaxel targeted with the antibody cetuximab that binds to epidermal growth factor receptor (EGFR), highly expressed on lung and breast tumors and found a drug uptake of 5.0% ID/g. However, no difference in drug delivery was found when the targeting antibody (cetuximab) was absent.

In summary, a large survey of delivery of therapeutic nanoparticles found an average delivery of only 2.23% ID/g to their targets after intravenous administration (Cheng et al., 2022). These delivery efficiencies are “quite low and represent a critical barrier in the clinical translation of nanomedicines”. It was “found that low delivery efficiency was associated with low distribution and permeability coefficients at the tumor site” (Cheng et al., 2022). Furthermore, “a recent meta-analysis revealed that active targeting typically increases the percent injected dose (% ID) by only 50% compared to passively targeted therapeutic nanoparticles, and clinical trials often show equivocal improvement” (Miller et al., 2017). These wide ranging studies highlight the fundamental problem of low delivery of therapeutic nanoparticles to solid tumors. Effective delivery of drugs to tumors is a fundamental unsolved problem. A recent report summarized the situation: “Typically, to deliver an intravenously injected nanomedicine to the cytosol of cancer cells in solid tumours it must overcome a series of biological barriers in a ‘CAPIR’ (circulation, accumulation, penetration, internalization and release) cascade, which includes (1) circulation in the blood compartments, (2) accumulation in the tumour, (3) deep penetration into avascular tumour tissue, (4) cellular internalization and (5) intracellular drug release. Among these barriers, nanomedicine infiltration into tumour tissue and to distal tumour cells from blood vessels remains an unresolved obstacle” (Zhou et al., 2019).

One widely used method for drug targeting is to bind the drug/molecule of interest to a receptor that has higher expression specifically at the target site compared to other regions. Frequently used for targeting are antibodies, peptides, and aptamers. For example, Herceptin is an antibody that targets highly expressed HER2 (Human Epidermal Receptor 2) protein on HER2-positive breast cancers tumors. Herceptin's high affinity to HER protein competes with growth factors, reducing growth factor binding and subsequent cell growth signal cascades.

Most targeting strategies have these limitations: 1) the highly expressed protein or receptor in the target tissue is usually expressed elsewhere in the body, competing with and limiting specific delivery; 2) the highly expressed binding sites of interest are often on tumor or other targeted cells which are not directly exposed to the blood and consequently are unable to efficiently bind to intravenously injected “targeted” drug/materials. The tumor or target cells are therefore behind a ‘firewall’ of endothelial cells, other cells, and extracellular matrix proteins that limit exposure to the nanoparticle which is in the bloodstream; 3) injectates have limited blood half-lives and are cleared by the kidneys and/or the reticuloendothelial system (RES, or mononuclear phagocyte system, MPS), especially by the liver and spleen. Uptake into the tumor requires many circulatory passes to allow diffusion and/or transport into the tumor. Maximum tumor delivery typically takes about 24 hours (Andrew et al., 1990, Grunberg et al., 2005). Many drug delivery constructs are rapidly cleared from the blood, limiting tumor delivery; 4) The targeting moiety frequently competes with endogenous substances. For example, in the Herceptin case, the antibody competes with endogenous growth factor. In the case of RGD peptide targeting to tumors, binding of the nanoparticle competes with endogenous RGD highly expressed in keratins which is prevalent in many tumors (Singh et al., 2017); 5) many drugs are hydrophobic, limiting their intravenous use to low levels; 6) the drug may be inactivated by blood enzymes, hydrolysis or other degradative processes before reaching the targeted site; 7) if a nanoparticle is used, the drug must be released into its active form; 8) further barriers must be overcome to achieve efficacious delivery, such as transcytosis, endocytosis, membrane diffusion, resistance to enzymes, resistance to drug efflux pumps (p-glycoproteins), and drug resistance; 9) targeting is frequently to a specific receptor on a particular tumor type which limits application to that type of tumor and may be thwarted by genetic variation or expression level of the epitope in various cells.

In summary, despite many attempts, the highly desirable targeting of medications has been difficult to fully achieve. However, the instant invention disclosed achieves significantly enhanced drug delivery.

Part of the treatment process disclosed involves vascular disruption. This has been reported as a therapy by itself using vascular disrupting agents (VDAs, reviewed by Smolarczyk et al., 2021). VDAs are tumor-specific, but so far have failed FDA approval, even though they caused necroses at tumor centers. At the periphery, tumor cells were found to acquire oxygen and nutrients from bordering non-tumor (and undamaged) vasculature resulting in continued tumor growth. Consequently, in a Phase III trial, patients did not have better survival with the VDA, so it was not approved. However, VDAs are currently being further investigated in combination with drugs since the VDAs provide a time window within which the vasculature is more leaky allowing better drug penetration. Drugs under study include: anti-angiogenic drugs, hypoxia inhibitors, chemotherapy, radiotherapy, and immunostimulatory agents (Smolarczyk et al., 2021). However, a recent Phase II trial showed little patient benefit when combining the vascular disrupting agent CA4P (fosbretabulin tromethamine) with carboplatin, paclitaxel, and bevacizumab for advanced non-squamous non-small-cell lung cancer (Garon et al., 2016).

### BRIEF SUMMARY OF THE INVENTION

In certain aspects, provided herein is a composition comprising nanoparticles comprising fibrinogen or a functional fragment thereof and a hydrophobic chemotherapeutic agent, wherein the average diameter of the nanoparticles is less than about 5 μm.

In certain aspects, provided herein is a composition comprising a hydrophobic chemotherapeutic agent, a protein, and a component having affinity for activated platelets that induces aggregates in serum at least 10 μm in size, wherein the average diameter of the nanoparticles is less than about 5 μm.

In some embodiments according to any one of the compositions described above, the component having affinity for activated platelets is fibrinogen or a functional fragment thereof.

In some embodiments according to any one of the compositions described above, the fibrinogen and the chemotherapeutic agent are distributed throughout the nanoparticles. In some embodiments, the fibrinogen and the hydrophobic chemotherapeutic agent are present on the surface of the nanoparticles. In some embodiments, the fibrinogen and the hydrophobic chemotherapeutic agent are non-covalently associated in the nanoparticles. In some embodiments, the ratio of fibrinogen to hydrophobic chemotherapeutic agent in the composition is about 20:1 to about 2:1. In some embodiments, the ratio of fibrinogen to hydrophobic chemotherapeutic agent in the composition is to about 12:1 to about 4:1.

In some embodiments according to any one of the compositions described above, the nanoparticles have a homogeneous structure. In some embodiments, the nanoparticles further comprise a carrier protein. In some embodiments, the carrier protein is albumin or transferrin. In some embodiments, the hydrophobic chemotherapeutic agent is selected from the group consisting of paclitaxel, camptothecin, docetaxel, and artemisinin. In some embodiments, the nanoparticles further comprise an antibody that binds to a protein located on the surface of platelets. In some embodiments, the average diameter of the nanoparticles in the composition is about 110 nm to about 400 nm. In some embodiments, the composition does not comprise a denaturant. In some embodiments, the nanoparticle further comprises a second chemotherapeutic agent.

In some embodiments according to any one of the compositions described above, the composition further comprises a pharmaceutically acceptable excipient or buffer. In some embodiments, the composition is a pharmaceutical composition. In some embodiments, the composition is sterile.

In certain aspects, provided herein is a method of treating a solid tumor in an individual comprising administering i) a treatment causing vascular damage and ii) the composition described herein to the individual.

In certain aspects, provided herein is a method of creating embolism in tumors comprising administering the composition described herein, wherein the nanoparticles are digested with the proteolytic enzymes released by necrotic cells, thereby releasing the drug.

In some embodiments according to any one of the methods described above, the coagulation cascade is activated. In some embodiments, a clot is formed in the tumor vasculature. In some embodiments, the nanoparticles bind to activated platelets. In some embodiments, each nanoparticle binds to two or more platelets.

In some embodiments according to any one of the methods described above, the treatment causing vascular damage is selected from the group consisting of administering a vascular disrupting agent, applying radiation, X-rays, microwaves, infrared, radio frequencies, heat, ultrasound, mechanical insult, or antibody-drug conjugates that are targeted to the solid tumor. In some embodiments, the treatment causing vascular damage is selected from the group consisting of DMXAA, CA4P, Plinabulin, CKD-516, AVE8062, AVE9062 OXi4503, MPC6827, BNC105P, ABT-751, VEGF-gelonin, Verubulin, and flavone-8-acetic acid (FAA).

In some embodiments according to any one of the methods described above, the nanoparticles preferentially localize to the site of the solid tumor. In some embodiments, the nanoparticles form aggregates at the solid tumor site. In some embodiments, the aggregates are at least about 1 μm in size. In some embodiments, the hydrophobic chemotherapeutic agent is released at the site of the solid tumor. In some embodiments, the treatment causes cell lysis of cells within the tumor. In some embodiments, the treatment causes selective damage of endothelium associated with the solid tumor. In some embodiments, the hydrophobic chemotherapeutic agent is released from the nanoparticles by an active release process. In some embodiments, the hydrophobic chemotherapeutic agent is released from the nanoparticles by a passive release process. In some embodiments, an immune response is stimulated at the solid tumor.

In some embodiments according to any one of the methods described above, greater than about 10 mg/kg of fibrinogen is administered to the individual in the form of nanoparticles. In some embodiments, about 10 mg/kg to about 500 mg/kg of fibrinogen is administered to the individual in the form of nanoparticles. In some embodiments, about 350 mg/kg of fibrinogen is administered to the individual.

In some embodiments according to any one of the methods described above, the treatment does not cause prohibitive cell lysis or damage at normal tissue and/or wherein the hydrophobic chemotherapeutic agent is not prohibitively released at normal tissue.

In some embodiments according to any one of the methods described above, the hydrophobic chemotherapeutic agent is administered at a higher level in the nanoparticle composition than the maximum tolerated dose for the hydrophobic chemotherapeutic agent administered in a non-nanoparticle formulation. In some embodiments, the hydrophobic chemotherapeutic agent is paclitaxel, and wherein 30 mg/kg to 90 mg/kg paclitaxel is administered to the individual in the form of nanoparticles.

In some embodiments according to any one of the methods described above, the vascular damaging agent or process is administered prior to, simultaneously with, or after the composition comprising nanoparticles. In some embodiments, the composition comprising nanoparticles is administered intravenously, intratumorally, or intraperitoneally. In some embodiments, the treatment causing vascular damage is administered intravenously, intratumorally, or intraperitoneally.

In some embodiments according to any one of the methods described above, the solid tumor is selected from the group consisting of lung and bronchus, breast, prostate, colon, rectal, melanoma, bladder, kidney, endometrial, pancreatic, thyroid, liver, intrahepatic bile duct, gastrointestinal, brain and nervous system, cervical, head and neck, ovarian, testicular, eye, skin, lymphomas and bone and muscle sarcomas.

In some aspects, provided herein is a kit comprising the composition described herein and a vascular disrupting agent.

In some aspects, provided herein is a method of producing the composition described herein, comprising sonicating the fibrinogen and the hydrophobic chemotherapeutic agent to produce nanoparticles. In some embodiments, the method described herein further comprises, prior to the sonication, dissolving fibrinogen in a buffered solution and heating the dissolved fibrinogen at 37° C. for at least 5 minutes.

### BRIEF DESCRIPTION OF THE DRAWINGS

FIG. 1. General schematic structure of one embodiment of the invention, the nanoparticle. A (white circles) points to fibrinogen molecules or other activated platelet binding material. B (small black dots) points to a drug or other payload or hydrophobic substance. C (grey circles) points to another optional protein or other included material. The minimal construct includes only A and B, but other disclosed constructs include any number of additional proteins or substances and any number of drugs or other payload materials.

FIG. 2. General schematic nanoparticle structure of another embodiment of the invention. This is a central cross-section view. A (white circles) points to fibrinogen molecules or other activated platelet binding material. B (small black dots) points to a drug or other payload or hydrophobic substance. C (grey circles) points to another protein or other included material. The minimal construct includes only A and B, but other disclosed constructs include any number of additional proteins or substances and any number of drugs or other payload materials.

FIG. 3. Clauss clotting assay. This compares the clotting times of free fibrinogen with the fibrinogen-drug nanoparticles. The nanoparticles clotted at a much higher rate than fibrinogen by itself. The average blood concentration of fibrinogen is 2 mg/mL and this is compared to a treatment fibrinogen nanoparticle concentration of 4 mg/mL. At these comparative levels, the fibrinogen nanoparticles caused coagulation at a rate 30 times that of endogenous fibrinogen.

FIG. 4. Shows tumor treatment progression over time of a subcutaneous tumor growing in the leg of a mouse.

FIG. 5. Shows tumor treatment progression over time of a subcutaneous tumor growing in the leg of a mouse.

FIG. 6. Shows tumor treatment progression over time of a subcutaneous tumor growing in the leg of a mouse.

FIG. 7. Shows tumor treatment progression over time of a subcutaneous tumor growing in the leg of a mouse.

FIG. 8. Targeting ear vascular damage in vivo with fibrinogen-oil red o nanoparticles compared to albumin-oil red o nanoparticles. The arrow points to accumulation of fibrinogen-oil red o nanoparticles at the site of vascular damage.

FIG. 9A depicts images of fibrinogen-paclitaxel nanoparticles in PBS or fetal calf serum at different lengths of time (30 seconds, 1 min, and 12 min).

FIG. 9B depicts a graph plotting the average size of aggregates (that were greater than 10 μm) over time.

FIG. 9C depicts a graph plotting the size of fibrinogen-paclitaxel nanoparticles in PBS over time.

FIGS. 10A-10D depicts a schematic of the steps of the treatment process at the molecular level.

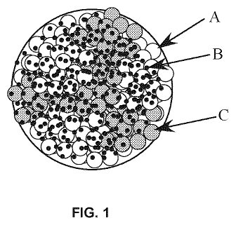

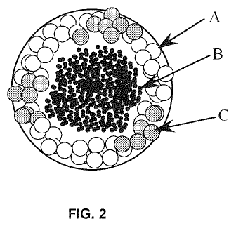

### DETAILED DESCRIPTION OF THE INVENTION

### I. Definitions

In general, terms used in the claims and the specification are intended to be construed as having the plain meaning understood by a person of ordinary skill in the art. Certain terms are defined below to provide additional clarity. In case of conflict between the plain meaning and the provided definitions, the provided definitions are to be used.

The term “about” as used herein refers to the usual error range for the respective value readily known in this technical field. Reference to “about” a value or parameter herein includes (and describes) variations that are directed to that value or parameter per se. For example, description referring to “about X” includes description of “X.”

As used herein “treatment” is an approach for obtaining beneficial or desired results. For purposes of this invention, beneficial or desired results include, but are not limited to, any one or more of: alleviation of one or more symptoms, diminishment of extent of disease, stabilized (i.e., not worsening) state of disease, slowing of disease progression and amelioration of the disease state. The methods of the invention contemplate any one or more of these aspects of treatment.

“Comprising” is intended to mean that the compositions and methods include the recited elements, but not excluding others. “Consisting essentially of” when used to define compositions and methods, shall mean excluding other elements of any essential significance to the combination. For example, a method consisting essentially of the elements as defined herein would not exclude other elements that do not materially affect the basic and novel characteristic(s) of the claimed invention. “Consisting of” shall mean excluding more than trace amount of, e.g., other ingredients and substantial method steps recited. Embodiments defined by each of these transition terms are within the scope of this invention.

The phrase “component having affinity for activated platelets” as used herein can be fibrinogen, functional fibrinogen fragment, fibrinogen variant, or molecule that binds similarly.

The term “functional fragment” as used herein is a fragment of fibrinogen that can bind to activated platelets.

### II. Delivery Process

The disclosed delivery process comprises two steps. Step 1 is to selectively damage tumor endothelium. Step 2 is to bind a nanoparticle construct to activated platelets that accumulates at the damaged tumor region.

First, tumor blood vessels are damaged, exposing molecules and substances that are normally hidden by the intact endothelial lining of the vessels. These newly exposed substances (such as tissue factor, collagen, thrombin, and ADP) activate platelets which bind to the damaged vessel. The activated platelets express a binding site on their surface, GPIIb/IIIa which binds fibrinogen. Circulating fibrinogen links to the platelets, but because fibrinogen is a dimer it links two platelets together and this crosslinking solidifies the platelets into a platelet thrombus. It is at this step where the disclosed nanoparticle construct intervenes. One of the components of the nanoparticle construct is fibrinogen itself or peptides, antibodies, antibody fragments, aptamers, other substances or molecules that bind to activated platelets. These components allow the nanoparticles to become incorporated into the thrombus. The nanoparticle size is larger than a single fibrinogen and when bound can more rapidly expand the thrombus to larger dimensions which can then occlude the blood vessel more effectively, cutting off the blood supply to downstream tumor cells, thus starving them of oxygen and nutrients, causing their demise. The nanoparticle also contains one or more drugs. Once bound to activated platelets, the drug containing nanoparticles release their drug contents in the tumor vicinity. The drug release mechanism can be passive or active. Passive release can be enhanced by non-covalent attachment to the nanoparticle construct. For example, it is disclosed that hydrophobic drugs or substances can be made to bind to various proteins to form nanoparticles. Active drug release can be achieved in several ways such as bonds that break in low pH environments (e.g., imines) since tumor environments are acidic due to the higher metabolism and poor blood flow. Tumors and tumor endothelium also express certain enzymes, such as metalloproteinases and γ-glutamyl transpeptidases, and these may be used to break bonds specific for these enzymes to release the drugs. The combination of vascular disruption, platelet binding molecules and drugs overcomes the shortcomings of many other targeting schemes. For example, vascular disrupting agents (VDAs) have been used to cause vascular collapse and demise of downstream tumor cells. However, tumor cells near the periphery were found to survive since they can get their oxygen and nutrients from non-tumor blood vessels at the tumor periphery. Although VDAs have been combined with drug treatments, the effects are not fully effective due to the toxic limits of drug administration. In those trials, free drug is used and not combined with the platelet-binding nanoparticle which can greatly increase delivery amount, specificity, and reduce off-target delivery or clearance. By combining the drug directly in the activated platelet targeting nanoparticle, much more of the drug is delivered to the tumor region allowing higher amounts to be administered thus obtaining superior treatment outcomes.

Surprisingly, this approach overcomes many drawbacks of conventional targeting processes discussed above. When tumor endothelium is damaged, the body responds by activating platelets that are then crosslinked to form a sort of “band-aid” or patch covering the lesion. Activation of the platelets causes the glycoprotein GPIIb/IIIa (the integrin α_{IIb}β₃) to be added to their surface and be switched to an active form. This then binds fibrinogen which is a dimer and can link two platelets together, yielding a platelet aggregate or thrombus. However, during this process, by placing in circulation a nanoparticle containing fibrinogen (or other activated platelet binder, e.g., proteins, peptides, antibodies, aptamers), the nanoparticles integrate into a forming platelet aggregate. Nanoparticles have multiple binding sites per particle, and therefore can participate in platelet crosslinking. This overcomes one of the major drug delivery problems because the activated platelets are exposed to circulating blood containing the nanoparticles. There are no ‘firewalls’ to overcome. The nanoparticles do not have to cross tumor endothelium and traverse through extracellular matrix to reach target sites on tumor cells. Another major advantage is that the binding to activated platelets is an extremely rapid and efficient process, perfected by evolution, since it involves the normal clotting process in wound response and repair and is essential for preservation of life when blood vessels are damaged. Without this process, small wounds would not stop bleeding, resulting in death. Another advantage is that large payloads of drug, imaging agent, or other substance can be carried by the nanoparticle compared to small molecule drugs by themselves or in conjugates. A further advantage is that the nanoparticles can be larger than typically required for nanoparticle drugs. Conventional wisdom and studies conclude that nanoparticle drugs should optimally be 60-80 nm (Perrault et al., 2009) with an effective therapeutic window between 50 and 200 nm (Madani et al., 2018, Kong et al., 2000). However, in the process disclosed, much larger nanoparticles are useful, even up to 5 microns in size. The capillary size is 5-10 microns and platelets are 2 microns. The nanoparticle-drug construct disclosed does not have to penetrate into tumors via the leaky tumor endothelium (the Enhanced Permeability and Retention, EPR effect), but can just be ‘caught’ by activated platelets in the blood vessels. Although a small size (<200 nm) can be used for tumor penetration, a larger size may also be favorable in order to accentuate the tumor treatment by creating larger and more rapid nanoparticle-platelet thrombi, thus embolizing the blood vessels causing an infarction, thus cutting off oxygen and nutrients leading to tumor cell death. Another advantage is that the target for the nanoparticles (the activated platelets) is extremely accessible to blood borne particles, resulting in many more of the nanoparticles being captured leading to higher delivery (higher % ID/g) and leaving less for off-target accumulation, thus achieving better specificity and efficiency. Another advantage is that the targeting process is not dependent on a particular tumor type or expression of a specific protein or receptor, but builds on the clotting/wound repair system, thus making the process applicable to a wide range, and perhaps all, solid tumor types.

In addition to cancer, this composition and process may also be useful in other cases for treating unwanted growths such as atherosclerosis in arteries in the heart, neck, brain, and other areas. Active plaque development is accompanied by angiogenesis to accommodate new growth. Angiogenic endothelium, as in tumors, is preferentially damaged by vascular disrupting agents, radiation, mechanical, and other stresses. Platelets then respond to this damage and become activated. This then provides priming of the region with activated platelets that will bind the nanoparticles targeted with fibrinogen or functional fibrinogen fragments to cause destruction of the aberrant tissue by vascular embolism and drug delivery.

### III. Platelet Activation

The first step in the process is to create vascular damage at the cancer site or other location where it is desired to target nanoparticles. This damage initiates a normal body response of activating platelets that aggregate at the damage site. In more detail, platelets are exquisite sensors of vascular damage and form an amplified target for the nanoparticle-drug construct. Platelets sense and can be activated by altered blood flow and mechanical stresses, but with endothelial damage they are activated by exposure to sub-endothelial materials including tissue factor, calcium, and ADP. Upon activation, platelets respond by intracellular calcium release from dense granules which causes alpha granule release of procoagulant and pro-inflammatory factors, membrane flipping (negative phospholipid translocation), and actin polymerization resulting in shape change (ellipsoid to irregular with many thin long spiny filopodia). The total membrane exterior surface becomes much larger, being supplied by fusion with the internal open canalicular system and dense tubular system as well as the alpha granule membranes. This greatly increases the surface area which magnifies the crosslinking ability for platelet aggregation. GPIIb/IIIa (the integrin α_{IIb}β₃) is the major receptor that is involved in aggregation. Under resting conditions, GPIIb/IIIa is not active, although approximately 50,000 receptors are present on the platelet surface. In the inactive form, GPIIb/IIIa is not available to mediate platelet-platelet bridging. However, upon platelet activation an additional approximately 20,000 copies of GPIIb/IIIa become expressed on the cell membrane (primarily through the exteriorization of the open canalicular system and the fusion of α-granules with the cell membrane) and all of the GPIIb/IIIa receptors become active and able to mediate platelet-platelet adhesions. As with many integrin receptors, the presence of calcium increases the affinity of the GPIIb/IIIa for its ligand. Inhibitors of GPIIb/IIIa (e.g., abciximab, trade name ReoPro) are used clinically to prevent thrombus formation. The key molecule that binds to GPIIb/IIIa is fibrinogen. It is a large dimeric protein and each monomer is composed of three different protein chains: Aα, Bβ, and γ. It is somewhat stick shaped, having binding sites at both ends, thus enabling linking between GPIIb/IIIa receptors on adjacent platelets, bridging the two: GPIIb/IIIa-fibrinogen-GPIIb/IIIa. Each fibrinogen molecule possesses three pairs of potential platelet-binding peptide sequences, two RGD sequences in each of the Aα chains (Aα 95-96 and Aα 572-674) and a dodecapeptide sequence, γ₁₂ (γ 400-411, HHLGGAKQAGDV (SEQ ID NO: 1)) in each of the carboxyl-termini of the γ chains (γC). Many other studies have used RGD sequences for targeting, but these often result in minimal incremental localization of nanoparticles. Here we disclose using the more extensive γ₁₂ sequence or fibrinogen itself to target activated platelets. In fact it has been shown that the γ₁₂ sequence is the most important for platelet adhesion and deletion of the RGD sequences showed no impact on fibrinogen's ability to induce platelet adhesion. The use of fibrinogen for targeting has been evolutionarily perfected to work only with activated platelets, otherwise massive fatal thromboses would occur. Other activated platelet binders may also be used including fibrinogen peptide fragments, aptamers, and antibodies that bind to GPIIb/IIIa, collagen, and von willebrand factor.

Activation of platelets by vascular damage in its completeness is a complex process with many factors and proteins involved. The result is that the discoidal platelets change to irregular shapes with many thin filipodia with much increased total membrane surface area. The proteins and lipid surface composition change dramatically. This natural process is a rapid and extensive change that amplifies the even slight or minor initiating event. It is disclosed herein how to harness this incredible process to target and treat cancer and other conditions. Most other targeting schemes depend on specific proteins expressed on particular cancers. For example prostate membrane specific antigen (PMSA) is used to target nanoparticles to prostate cancer. But here we teach away from this logic and target a process that can be initiated in almost all tumor types, for example, prostate cancers, breast cancers, lung cancer, and pancreatic cancers. It has been noted that triple negative breast cancer (TNBC) is more difficult to treat since it lacks the three targetable receptors found on other breast cancers: estrogen, progesterone, and HER2 receptors. However, here we instead target damaged endothelium that can be created in all solid tumors.

### IV. Treatments Causing Vascular Damage

There are a number of methods that can be used to cause vascular damage. The damage should be specific for the indication such as cancer. Vascular disrupting agents have been found that create specific tumor vascular damage, including DMXAA, CA4P, Plinabulin, CKD-516, AVE8062, AVE9062 OXi4503, MPC6827, BNC105P, ABT-751, VEGF-gelonin, Verubulin, flavone-8-acetic acid (FAA) among others (Smolarczyk et al., 2021, Mita et al., 2013, Cai et al., 2006, Porcu et al., 2014).

Tumor-specific antigens have been used to target other materials to tumors (e.g., cellular toxins) that can then damage tumor vasculature. Vascular targets include proteins expressed on tumor endothelial cells, such as DELTA4, ROBO4, endosialin, TEM5, TEM8, epidermal growth factor receptors, as well as those that are secreted into the stroma around the vessels, such as the differentially spliced isoforms of fibronectin, tenascin (neri05), and collagen. Ligands for the vascular targets include antibodies, peptides, small organic molecules, and aptamers.

Tumors can also be identified by their higher metabolism and targeted by e.g., glucose, their lower pH, their hypoxic state, and enzymes. Many tumors secrete metalloproteinases and other enzymes that can be used for targeting. The tumor target can then be used to bring substances such as drugs or other materials to enable specific vascular damage.

Heat may be used to damage vasculature. For example, absorptive gold nanoparticles or melanin can be irradiated with light or infrared radiation to heat the tumors causing vascular damage (photothermal therapy). In fact, tumor endothelium is more sensitive to heat than normal tissue endothelium, so heat alone can be used to specifically damage tumor vessels (Song et al., 1980, Fajardo and Prionas, 1994). Heat can be applied by thermal generators such as light bulbs, infrared lamps, electric heating of wires or other resistive material, microwaves, ultrasound, induction heating, magnetic nanoparticle hyperthermia, exposure to alternating magnetic fields, radiofrequency, hot air introduced into the lungs, the abdomen, or other body regions, and warming the blood.

Vascular damage can be created mechanically. For example, tumors can be recognized by MRI, CT, or other imaging means for directing the mechanical or heating generator, such as focused ultrasound to cause specific vascular and tissue disruption.

Radiation can damage endothelium. It is known that tumor endothelium is more sensitive to radiation than normal tissue, so radiation can be used to specifically damage tumor vessels (Song et al., 2014). The radiation dose needed is mostly in the range of 5 to 20 Gy and with a very potent effect at about 12 Gy. These levels are well tolerated in virtually all body regions making this approach feasible for almost all cancers. For example, lung cancer kills more people than any other cancer. By the time it is detected it is more advanced, stage 3. However, although it is spread with lesions all over the lungs, it is typically still confined in the chest cavity, not yet having metastasized to other parts of the body. Using radiation, a dose delivered to this region can cause specific vascular damage in the tumor lesions. However a curative dose is not possible due to ancillary normal tissue damage. Another difficult cancer to control is ovarian where the tumor growths are confined to the peritoneal cavity at usual time of diagnosis. However, the lesions are spread all throughout. Low level radiation can be used to ‘paint’ the tumors for destruction. Even if tumors have metastasized, radiation can be used in multiple regions to create specific targets for the drug. Radiation use to damage tumor vasculature or specific sites is attractive since it can be confined to regions.

Combining vascular damaging approaches can both be additive and synergistic to reach better therapeutic effects. For example, heat applied 1-6 hours after VDA treatment was found to be synergistic in creating specific tumor vascular damage (Horsman, 2006).

The therapy is then in the next step targeted to these damage sites. It is akin to a laser ‘painting’ a target to guide a missile attack. The specific tumor vascular damaging is important since it overcomes the major problems with surgery which are: a) some tumor is left behind, b) tumor is spread and close to sensitive tissues and may be inoperable, c) adjacent important normal tissue is damaged. Our method may better confine the damage and targeted drug delivery in the next step.

### V. Nanoparticle Targeting

In more detail the second step in the disclosed process is introduction of a nanoparticle that binds to the activated platelets which result from damaged endothelium or vascular damage. Fibrinogen on a nanoparticle may be used for this purpose due to its active and selective binding to activated platelets. Since it is particular sequences, RGD and HHLGGAKQAGDV (SEQ ID NO: 1), that bind to the activated platelet GPIIb/IIIa, these or similar sequences may be used. Other proteins may be used that also bind activated platelets, namely von Willebrand factor (vWF), fibronectin, thrombin, collagen, and vitronectin. Peptide sequences from these proteins or fragment variants may also be employed. Binding may also be achieved by using antibodies, antibody fragments, aptamers or various chemicals or compounds that have affinity to activated platelets. By incorporating multiple copies of fibrinogen or other binding moieties in the nanoparticle, a higher binding constant is achieved due to multipoint attachment. Binding goes up exponentially with number of binding sites. This can facilitate higher binding than obtained with endogenous plasma single molecule fibrinogen, and out-compete it for binding to the platelets. A further advantage of this process is that strong binding of the nanoparticle stimulates “outside-in” signaling of the platelets. Fibrinogen binding induces stabilization of platelet adhesion, platelet spreading, granule secretion and amplification of platelet aggregation leading to growth to a potentially occlusive intravascular thrombus. This process then not only builds on a normal amplified body response but stimulates further amplification by avid binding to the platelets. There are then two amplification processes: the first when platelets respond to vascular damage and a second stimulated by binding of the nanoparticle to the activated platelets. It is here disclosed the composition and process of a nanoparticle to participate in this sequence of events and the benefits thereof. One significant benefit is that a large and accessible target is provided by the activated and aggregating platelets. There are a huge number of binding sites that become exposed just at the vascular damage site. This gives great specificity to the nanoparticle to bind there in great preference to other benign sites in the body. This is important for drug delivery, making it very specific. Another aspect is that for intravenously injected nanoparticles, the target site is in the vasculature and there is no need to cross endothelial barriers, extracellular matrix barriers, and tumor membrane barriers. The platelets are in the vasculature and directly presented to the intravascular nanoparticles. All of this adds up to greatly enhanced targeting of the nanoparticles.

The present invention reflects a significant advance in treatment of solid tumors. For example over 2 years of research, treatments showed no clear infarctions leading to clear necroses, even though varying amounts of all components were tried. One of the components, fibrinogen, was varied from 10 to 150 mg/kg. However, when 300 mg/kg was tested, surprisingly, massive necroses rapidly developed in the tumors within a day. Another example is propidium iodine, which is an excellent binder to DNA in necroses, and was tested for inclusion in a nanoparticle for targeting necroses. However, this failed under the conditions used, possibly due to instability in retention in the nanoparticle. Surprisingly, a weaker DNA binder, acridine yellow G incorporated into the nanoparticles was found to target the nanoparticles to free DNA, which is released upon cell death.

Another surprising finding was that paclitaxel (PTX) at 90 mg/kg was well-tolerated when delivered in the nanoparticles provided herein. This is considerably higher than the reported maximum tolerated dose of 10 mg/kg for this drug. The fact that more could be safely administered might be explained by better targeting, leaving lower amounts to cause off-target toxicities.

An additional surprising finding was that nanoparticles made with camptothecin (CPT 5.4 mg/kg) and fibrinogen (300 mg/kg) were typically larger than 200 nm. However when 120 mg/kg of transferrin was included, it was possible to obtain nanoparticles smaller than 200 nm. What was found was that fibrinogen which is a rather “sticky” protein could be moderated by combination with other proteins in the nanoparticle such as transferrin or albumin. In some embodiments, nanoparticle size impacts blood residence time, toxicity and efficacy. In a preferred embodiment, 60 mg PTX/kg and 350 mg fibrinogen/kg are formed into nanoparticles 110-180 nm or 110 to 350 nm in size. Another discovery process requiring extensive experimentation was to find the type of fibrinogen that worked well. A number of different fibrinogens exist in the blood, since some forms are missing various peptides or bound components such as plasminogen, von Willebrand factor, thrombospondin, and other substances. Isoforms of the component chains exist, such as Aα and AαE, γ and γ′, and as a glycoprotein, molecules can contain varying amounts of carbohydrate (usually 4-10% w/w). Various forms and fractions were tested to find a preferable one for tumor targeting. Surprisingly, a less pure form, that without removal of tightly bound plasminogen, formed favorable nanoparticles and bound the drug paclitaxel in high amounts, forming <200 nm nanoparticles. These worked well for embolizing damaged tumor capillaries. This is counter-intuitive since plasminogen converts to plasmin which inhibits and disassembles clots. Additional experiments showed that gently dissolving very pure human fibrinogen depleted of plasminogen could also produce equivalent ‘curative’ in vivo results using a modified preparation method for the nanoparticles.

In some embodiments the nanoparticles induce aggregation. In some embodiments, the aggregation is aggregation of platelets. In some embodiments, the nanoparticles further comprise a component having affinity for activated platelets that induce the formation of aggregates. In some embodiments, the nanoparticles induce aggregation within at least 1 second, at least 2 seconds, at least 3 seconds, at least 4 seconds, at least 5 seconds, at least 10 seconds, at least 20 seconds, at least 30 seconds, at least 40 seconds, at least 50 seconds, at least 1 min, at least 2 min, at least 4 min, at least 6 min, at least 8 min, at least 10 min, at least 12 min, at least 14 min, at least 16 min, at least 18 min, at least 20 min, at least 22 min, at least 24 min, at least 26 min, at least 28 min, or at least 30 min of mixing with serum or albumin. In some embodiments, the aggregates are at least 5 μm, at least 10 μm, at least 15 μm, at least 20 μm, at least 25 μm, or at least 30 μm in size. In some embodiments, the aggregates are loosely and non-covalently linked. In some embodiments, the aggregates accelerate the blockage of blood vessels. In some embodiments, the aggregates bind to activated platelets. In some embodiments, the aggregates become crosslinked. In some embodiments, the aggregates contribute to more effective embolization. In some embodiments, the nanoparticles are stable in serum without causing serum aggregation.

### VI. Novel Drug Release

Provided herein is a novel method to greatly enhance the delivery and release of drug to a tumor. In some embodiments, the method comprises creating necrosis in a tumor which then releases proteolytic enzymes from the dead cells which in turn breaks down the protein-drug nanoparticle, releasing the drug.

In a preferred embodiment, vascular damage is created using a vascular disrupting agent. In some embodiments, the vascular disrupting agent may be administered systemically to a subject. In some embodiments, the vascular disrupting agent may be administered locally to a subject. In some embodiments, the vascular disrupting agent leads to platelet activation and platelet crosslinking by a fibrinogen-drug nanoparticle. In some embodiments, the thrombi formed embolizes tumor vessels. In some embodiments, the embolization causes tumor cell death and release of cellular proteolytic enzymes. In some embodiments, the released cellular proteolytic enzymes degrade the nanoparticle-protein-drug nanoparticle, which releases the drug.

This method yields a number of substantial advantages over other drug delivery and release approaches. One advantage is that the nanoparticles amplify the coagulation process that is initiated by the vascular disrupting agent. Having multiple interacting sites on the nanoparticle that interact with activated platelets causes the coagulation process to be amplified resulting in an extensive progression of the process, stimulating more platelets to be activated and providing more interacting sites than results from a normal coagulation response. A more extensive embolization is achieved, depleting downstream tumor cells of oxygen and nutrients, resulting in massive cell death and release of large amounts of proteolytic enzymes. Current mechanisms for enzyme release rely on low levels of endogenous enzymes secreted by tumor cells. The mechanism of the methods provided herein is advantageous in that the quantity of released enzymes is significantly higher than the endogenous release method, leading to more effective drug delivery from nanoparticles. Since the nanoparticle contains one or more drugs, an advantage is the massive drug delivery in the tumor region after the release of proteolytic enzymes, which digest the nanoparticles resulting in drug delivery.

Previous use of vascular disrupting agents (VDAs) has generally been disappointing with the result that some central tumor cells die due to vascular flow reduction, but peripheral tumor cells still obtain oxygen and nutrients from blood vessels unaffected by the VDA. These cells regrow the tumor and result in little overall patient gain. DMXAA, a promising VDA, was rejected by the FDA in Phase III trials due to lack of patient gain in survival. In this disclosure, drugs are massively released to access and eradicate the surrounding tumor cells.

A further advantage of the methods and compositions provided herein is stimulation of the immune system. When tumor cells die, their membranes break down and internal contents become exposed, presenting new antigens to the immune system which can generate a new tumor-specific immune response. This immune response is known not only to affect the immediate tumor, but by the ‘abscopal effect’ attack tumors elsewhere, even distant, including metastases.

A further advantage of this method is reduced toxicity. In a preferred embodiment, the nanoparticle-protein-drug is stable and does not substantially break down in blood and does not release the drugs unless by the process disclosed above at the tumor, where proteolytic enzymes are produced. Because the presently claimed invention encases one or more drugs in nanoparticles, no free drug is in the general circulation which then avoids systemic toxicity and the toxicities usually incurred by antimitotic drugs, namely attack on the intestinal lining (of rapidly dividing epithelial cells) and the hematopoietic system in bone marrow where red cells and white (immune) cells are produced. These two systems largely limit the dose possible of drug administration, limiting the efficacy of many drugs to suboptimal levels. Furthermore, using a nanoparticle that does not release the drug except by proteolysis, redirects the drug that is not taken up by the tumor away from normal problem targets, like the intestines and bone marrow, and directs the nanoparticles largely to the liver where they can be safely metabolized and cleared. Nanoparticles in the 100-500 nm range are largely cleared by the liver and spleen, and are also imbibed by macrophages elsewhere. Once endocytosed by a macrophage (Kupffer cell in the liver), the nanoparticle will be broken down by enzymes in the lysosome. The released drug will be metabolized. For example, paclitaxel is metabolized by the cytochrome P450 system. Therapy with paclitaxel has not been clearly linked to cases of clinically apparent acute liver injury. Hence, one of the main problems with drug administration, that of off-target toxicity, can be greatly reduced by the presently claimed invention.

### VII. Nanoparticle Composition

A composition comprising nanoparticle is also provided herein. In some embodiments, the nanoparticle has a diameter of 15 nm to 5 microns, or 20 nm to 900 nm or the nanoparticles have an average diameter of 15 nm to 2 microns. In some embodiments the average diameter of the nanoparticles in the composition is less than about 5 μm, such as between about 110 nm and about 5 μm, between about 110 nm and about 2 μm, between about 110 nm and about 1 μm, between about 110 nm and about 800 nm, between about 110 nm and about 500 nm, between about 110 nm and about 400 nm, between about 110 nm and about 300 nm, between about 150 nm and about 500 nm, between about 150 nm and about 350 nm, or between about 150 nm and about 330 nm.

In some embodiments, the nanoparticle comprises fibrinogen and a hydrophobic chemotherapeutic agent. In some embodiments, the fibrinogen and the hydrophobic chemotherapeutic agent are distributed throughout the nanoparticle. In some embodiments, the fibrinogen and the hydrophobic chemotherapeutic agent are homogenously distributed throughout the nanoparticle. In some embodiments, the nanoparticle does not comprise a shell. In some embodiments, the fibrinogen and the chemotherapeutic agent are present on the surface and the interior of the nanoparticle. In some embodiments, the nanoparticle comprises hydrophobic chemotherapeutic agent on its surface. In some embodiments, the nanoparticles, comprises exposed hydrophobic chemotherapeutic agent on its surface. In some embodiments the chemotherapeutic agent is “peppered” throughout the nanoparticle. In some embodiments, the hydrophobic chemotherapeutic agent is noncovalently associated with the fibrinogen in the nanoparticle. In some embodiments the hydrophobic chemotherapeutic agent is bound by fibrinogen. In some embodiments, the hydrophobic chemotherapeutic agent is bound by hydrophobic regions of the fibrinogen.

Without being bound by theory, in some embodiments, hydrophobic chemotherapeutic agent exposed on the surface of the particle interacts with serum proteins causing aggregation. These larger aggregates are then solidified by activated platelets (primed at the tumor site) and are large enough to embolize tumor vessels.

In some embodiments, the ratio of fibrinogen to chemotherapeutic agent in the composition is between about 20:1 to about 2:1, such as about 15:1 to about 2:1, about 14:1 to about 2:1, about 13:1 to about 2:1, about 12:1 to about 2:1, about 11:1 to about 2:1, or about 10:1 to about 2:1. In some embodiments, the ratio of fibrinogen to chemotherapeutic agent in the composition is between about 20:1 to about 4:1, such as about 15:1 to about 4:1, about 14:1 to about 4:1, about 13:1 to about 4:1, about 12:1 to about 4:1, about 11:1 to about 4:1, or about 10:1 to about 4:1. In some embodiments, the ratio of fibrinogen to chemotherapeutic agent in the composition is between about 12:1 to about 4:1, such as about 11:1 to about 4:1, about 10:1 to about 4:1, about 9:1 to about 4:1, about 8:1 to about 4:1, about 7:1 to about 4:1, or about 6:1 to about 4:1.

In some embodiments, the nanoparticle further comprises a carrier protein. In some embodiments, the carrier protein is a protein found in human serum. In some embodiments, the nanoparticle comprises albumin. In some embodiments, the nanoparticle comprises transferrin. In some embodiments, the nanoparticle comprises human serum albumin (HSA).

In some embodiments, the nanoparticle composition is sterile. In some embodiments, the nanoparticle composition is suitable for administration to a human. In some embodiments, the nanoparticle composition is a pharmaceutical composition. In some embodiments, the nanoparticle composition comprises one or more buffers or excipients. In some embodiments, the nanoparticle composition is suitable for intravenous administration. In some embodiments, the nanoparticle composition is suitable for intraperitoneal administration. In some embodiments, the nanoparticle composition is suitable for intratumoral administration.

In some embodiments, the nanoparticle comprises a binding moiety for activated platelets and this can include: fibrinogen, von Willebrand factor (vWF), fibronectin, thrombin, collagen, vitronectin. peptide sequences from these proteins, or other peptides that bind activated platelets. The binding moiety may also be antibodies, antibody fragments, aptamers or various chemicals or compounds that have affinity to activated platelets. Combinations may also be used. There are multiple copies (>1) of these binding moieties on the nanoparticle surface. This nanoparticle by itself can be utilized to enhance thrombus formation and vascular occlusion. The nanoparticle in addition in some embodiments carries a payload. The payload material can be a drug, imaging material, or other substance that is beneficial to be delivered to the activated platelet site. The payload may also be smaller nanoparticles, such as magnetic nanoparticles that can be used for imaging or hyperthermia treatments, for example. Drugs may include paclitaxel, docetaxel, camptothecin, artemisinin, cis-platin, and doxorubicin. Imaging payloads can include elements that can be detected by X-rays, elements or compounds that can be detected by MRI, e.g., containing gadolinium, radioisotopes, fluorescent molecules, magnetic resonance spectroscopy, functional magnetic resonance imaging, diffusion tensor imaging, materials detected by ultrasound, positron emission (PET), single photon imaging, magneto sensing, photoacoustics, infrared spectroscopy, second-harmonic imaging, bioluminescence, reflection, single photon emission, optical coherence tomography, and Raman imaging. The nanoparticles can contain iodine, radioactive iodine, yttrium, technecium, gold, and platinum.

The protein, proteins, or substances incorporated into the construct are chosen to have various activities. First is targeting to damaged vasculature, such as provided by in a preferred embodiment, fibrinogen. However, other proteins or substances can be included to, for example, change or reduce the size of the complex (e.g., albumin or transferrin), add additional targeting to tumor or other targets (e.g., transferrin, antibodies to tumor)

Various payloads can also be combined in the nanoparticle formation. In a preferred embodiment, paclitaxel is dissolved in polyethylene glycol 200 molecular weight and sonicated with fibrinogen to form nanoparticles that are less than 300 nm in phosphate buffered saline, pH 7.4. Other preferred constructs are transferrin-paclitaxel, fibrinogen combined with transferrin, fibrinogen plus camptothecin, fibrinogen and transferrin plus artemisinin. The latter is additionally effective since it brings a peroxide (artemisinin) and iron (in the transferrin) to the targeted site and these two can undergo (after reduction of the Fe+3 to Fe+2) the Fenton reaction creating free radicals that are very destructive to tissues. Many other hydrophobic drugs can be used in this process including other taxanes, docetaxel, epirubicin, hydrophobic doxorubicin, vinorelbine, vincristine, vinblastine, capecitabine, and poorly soluble antiviral drugs such as mangiferin and favipiravir.

The drug release, penetration, cell uptake, intracellular delivery, and preservation of activity are important considerations for efficacy. In one embodiment the disclosed process has the feature and advantage that the payload is only adsorbed to the nanoparticle. Because the drug incorporated is non-covalently bound or modified it can be released efficiently after nanoparticle targeting. It is known that small hydrophobic drugs easily pass directly through cell membranes, making their journey to the intracellular molecular targets efficient.

A more complete list of anticancer drugs that can be used includes: 4-methylumbelliferone, 9-aminocamptothecin, 5,6-dihydro-4H-benzo[de]quinoline-camptothecin, aclarubicin, actinomycin, amsacrine, bendamustine, bexarotene, betulin, bicalutamide, bleomycin, bortezomib, bosutinib, busulfan, cabazitaxel, cabozantinib, capecitabine, carboplatin, carmustine, chlorambucil, cisplatin, cladribine, cobimetinib, cyclopamine, cyclophosphamide, cytarabine, dacarbazine, dactinomycin, dasatinib, daunorubicin, decitabine, diaziquone, docetaxel, doxorubicin, enzalutamide, epirubicin, erlotinib, estramustine, etoposide, floxuridine, fludarabine, fluorouracil, fotemustine, hydroxyaniline mustard, hydroxyurea, gefitinib, gemcitabine, ibrutinib, idarubicin, ifosfamide, imatinib, irinotecan, ixabepilone, lapatinib, lenalidomide, letrozole, leucovorin, lomustine, mechlorethamine, melphalan, merbarone, mercaptopurine, mesna, methotrexate, mitomycin, mitoxantrone, mitozolomide, monomethyl auristatin, nedaplatin, nelarabine, nilotinib, nilutamide, N-nitroso-N-methylureaoxaliplatin, novobiocin, omacetaxine, paclitaxel, panobinostat, pazopanib, pemetrexed, pentostatin, plicamycin, pomalidomide, ponatinib, prednisolone, PR-104A, pyrrolo[2,1-c][1,4]benzodiazepine, quercetin, raltitrexed, regorafenib, romidepsin, ruxolitinibm, semustine, SN-38, sonidegib, sorafenib, streptozotocin, suberoylanilide hydroxamic acid, sunitinib, tamibarotene, tamoxifen, tarcedinaline, tegafur, temsirolimus, thiotepa, teniposide, topotecan, triptorelin, trabectedine, vandetanib, vemurafenib, venetoclax, vincristine, vinflunine, vinorelbine, vinblastine, vindesine, vismodegib, vorinostat, rifampin, rapamycin, idarubicin, and disulfiram.

In some embodiments, the nanoparticle comprises more than one chemotherapeutic agent, i.e. a hydrophobic chemotherapeutic agent and a second chemotherapeutic agent. In some embodiments, the second chemotherapeutic agent is hydrophobic. In some embodiments, the nanoparticle comprises paclitaxel and a second chemotherapeutic agent. In some embodiments, the nanoparticle comprises camptothecin and a second chemotherapeutic agent.

Other drugs or agents can also be used including antiviral drugs, gene therapy materials, and antibacterial drugs.

Disulfiram was developed as a treatment for alcoholism, but later found to help in stopping tumor growth and metastasis, and targets cancer stem cells and reduces tumor recurrence. However, disulfiram is not water soluble and difficult to target tumors. Here it is disclosed that it and other poorly water soluble drugs can be complexed with fibrinogen, transferrin, albumin, and other proteins by the methods disclosed that now enable high levels of the drugs to be delivered to tumors including metastatic lesions.

A fundamental novelty of this disclosure is the process of 1) damaging vasculature in the target region (which generates activated platelets), and 2) targeting activated platelets with a nanoparticle that binds activated platelets. Another fundamental novelty disclosed is the composition of the nanoparticle and process for its construction. In a preferred embodiment, the vasculature of cancer tissue is damaged by vascular disrupting agents, radiation, or heat, or combinations thereof, and a nanoparticle is made by sonicating fibrinogen with paclitaxel. Upon administration, the nanoparticle highly targets the activated platelets in the damaged region enhancing the occlusion of blood vessels leading to demise of downstream tumor cells, but also delivering a potent drug to tumor cells in the vicinity that may have otherwise survived due to nourishment and oxygen from peripheral patent blood vessels.

In a preferred embodiment, the treatment is composed of 3 components: 1) A material or procedure that damages blood vessels in the tumor. This can be done with a vascular disrupting agent, radiation, heat, microwaves, ultrasound, or other ways. 2) A nanoparticle containing fibrinogen or other substance that binds to activated platelets at the tumor created by the first component which enhances the clotting to the extent of major embolization of tumor blood vessels. 3) Deposition of a drug carried by the nanoparticle at the tumor. This strategy of building on the normal vascular damage response of the body takes advantage of the plentiful, and accessible binding sites generated by the body's amplification in response to vascular damage by attracting many platelets there and activating them. By also carrying a drug (or multiple drugs), the treatment beyond vascular embolism is greatly enhanced. The plentiful and extremely accessible platelet targets allow high efficiency of nanoparticle delivery and accumulation at the tumor, achieving drug delivery substantially higher than most other known drug delivery schemes. Two unique, novel and innovative aspects of this invention are: 1) Greatly enhanced tumor vessel embolism, resulting in rapid tumor destruction, and 2) greatly enhanced drug delivery, resulting in better drug treatment and fewer off-target toxicities.

In order to achieve these results, many experiments were carried out varying the amount of fibrinogen, use of transferrin in combination with fibrinogen for additional tumor targeting, using variable amounts of vascular disrupting agents, using various different vascular disrupting agents (e.g., DMXAA (Vadimezan, ASA404), CA4P (fosbretabulin tromethamine), and varying the amount and type of drug (paclitaxel (PTX), docetaxel, camptothecin, artemisinin, cis-platin, doxorubicin). For a long time no “spectacular” results were obtained, and tumors continued to grow. Sometimes some small part of the tumor would turn black, indicating necrosis. So by extensive variation of all these parameters over the course of 4 years, a formulation was arrived at that produced consistent tumor ablation. So by unobvious and extensive searching it was found that fibrinogen in the nanoparticle less than 200 mg/kg gave inferior tumor necrosis. A value of 350 mg/kg was found to give excellent tumor vessel embolization and necrosis. A value of DMXAA in excess of 20 mg/kg was found to lead to excessive toxicity. Current teaching, which is in contradiction to this, says that 27.5 mg/kg is useful, whereas less than 20 mg/kg is ineffective (Pedley et al, 1996). We found that 18 mg/kg was close to optimal. The amount of drug was also adjusted experimentally and found that values between 60-90 mg PTX/kg gave excellent results. This is in contradiction with the published maximum tolerated dose of PTX of 25 mg/kg (Bhattacharyya et al., 2015). It also emphasizes that using the disclosed method almost 4 times the maximum tolerated dose of drug can be safely administered. Coupled with substantially better targeting means the treatment at the tumor is greatly enhanced while reducing off-target accumulation and side effects.

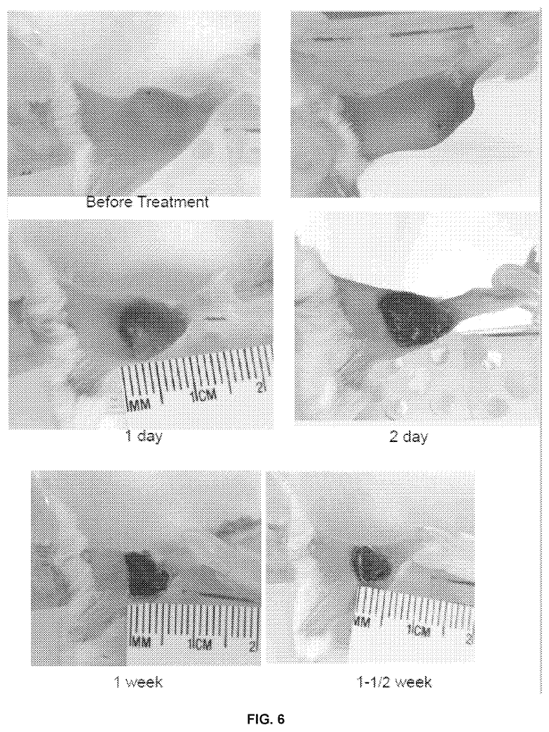

A surprising result was that tumors were completely eradicated with only a single quick treatment. Other cancer treatments typically require months, and often are not curative. Radiation treatments are typically daily for 6 weeks: drugs have to be administered for lengthy periods, or even indefinitely. Surgery is rather quick, but requires hospitalization, high cost, and lengthy recovery times. Even with these extensive treatments, cancers frequently recur. In a preferred embodiment, the fibrinogen-drug nanoparticle is administered intravenously (for at most 5 minutes) and 2 minutes later the vascular disrupting agent is injected intraperitoneally (for at most 2 minutes). In some embodiments, in under 10 minutes, the single treatment is administered. In animals we observe most of the tumor becomes necrotic in 1-2 days (see, e.g., FIG. 6) and the remainder is effectively killed by the novel drug delivery mechanism. Tumors shrink and by about 5 weeks are gone and do not return. Compared to other cancer treatments, the claimed invention is very surprising in that a single and simple 10 minute application can produce such profound and efficacious results.

Although a preferred embodiment is a nanoparticle constructed from fibrinogen containing a hydrophobic drug, a similar functionality and effective treatment can be achieved by other nanoparticle constructs. In another embodiment, the nanoparticle construct is a liposome with fibrinogen attached to its surface (or other activated-platelet targeting agent) and the drug contained within. If the drug is hydrophobic it can be included in the liposome membrane, or if hydrophilic, in the core space. Activated platelet-targeted liposomal doxorubicin is an example. Other nanoparticle constructs that can be made to bind to activated platelets and carry a payload may therefore be used in this disclosed process and include: dendrimers, polymersomes, PLGA, PLA, and similar polymer constructs, polymer micelles, nanospheres, silica nanoparticles, mesoporous silica nanoparticles, quantum dots, solid lipid nanoparticles, micelles, emulsions, metal nanoparticles, gold nanoparticles, magnetic nanoparticles, ferritin, carbon nanotubes, and carbon nanodiamonds.

Although much of what has been disclosed concerning targeting with the steps of vascular damage then binding to activated platelets, in another embodiment, hydrophobic drugs or substances are bound to various proteins or other components that have affinity to the target sites without invoking activated platelets. An example of this is transferrin complexed to the hydrophobic drug paclitaxel. Transferrin is of interest in tumor treatment or inflammatory regions since both endothelial cells and accelerated growth require iron which is normally provided by endogenous transferrin. By complexing with a drug, the drug is targeted to those sites. Additionally, with respect to brain tumors, brain endothelium has upregulated transferrin receptors and these provide enhanced transcytosis and transport through the usually impenetrable blood-brain-barrier. The method of complexing the drug as exemplified in the examples allows the drug to be released once brain tumor uptake occurs. Furthermore, free hydrophobic drugs can easily cross cell membranes thus delivering it into the cells where it can exert its therapeutic effects. In the case of paclitaxel, this binds to and stabilizes microtubules, halting cell division and triggering apoptosis.

It is here disclosed several further advantages of fibrinogen-drug nanoparticles: One is that the protein is biodegradable with favorable clearance. Once it performs its rather short lived (a few days) task of embolizing tumor blood vessels and delivering a drug it is no longer needed and can be degraded. A second additional advantage is that the drug is only complexed with the protein and can be released easily. This is compared to other nanoparticle drug carriers such as liposomes or polymers where the drug is tightly contained and often poorly released. A study of uptake by tumor cells of free doxorubicin compared to liposomal Dox showed cell uptake was 100 times lower with the liposomal Dox (Shahin et al., 2013).

### VIII. Necrosis Targeting

Despite the initial treatment which damages tumor endothelium and binds drug-carrying nanoparticles to activated platelets, some cancer cells may survive, causing recurrence. Thus, follow-up treatments may be necessary. In that case, additional treatment doses using the same method may be given, but may need to be used at lower doses due to several issues including the possibility of higher levels of circulating activated platelets that may cause the administered nanoparticles to aggregate more in the blood or other non-tumor areas possibly increasing their toxicity. A higher level of circulating activated platelets may be caused by higher levels of thrombin or exposed collagen, tissue factor, or other thrombogenic activating substances created by earlier treatments. To avoid adverse subsequent interactions, a second targeting method is disclosed: Nanoparticles that target necroses. The treatment using vascular damage and fibrinogen-binding-drug nanoparticles was shown to create massive cell death and extensive necrosis at the tumor. This now presents a new enormous tumor-specific target. To take advantage of this it is disclosed that nanoparticles carrying drugs are used that target necroses.

In one embodiment, the nanoparticles are constructed of a protein or substance (or multiple such materials) that binds necroses and incorporates one or more drugs. Biomarkers for necroses include: DNA/histone H1 complex, exposed DNA, heat shock protein 90 (Hsp90), lupus-associated (La) antigen, histones, high mobility group box 1 (HMGB1), fumarase and other unknown molecules. The nanoparticles disclosed will contain substances that bind to these markers that include: chTNT-1/B and NHS76 that bind the DNA/histone H1 complex, chTNT-3, TO-PRO-1, Hoechst 33258, Hyp, HAD, Shyp, Hypomycin A, Rhein, 1-hydroxyantha-quinone, Naphthazarin, Vitexin, ethidium bromide, propidium iodide, acridine orange, acridine yellow G, and other acridines, actinomycin D, doxorubicin, daunomycin and other anthracyclines, ellipticine, cisplatin, bleomycin, berberine, proflavine, DAPI, and thalidomide that binds exposed DNA, GSAO that binds Hsp90, the antibody DAB4 that binds La antigen, Glucarate, Heparin, Chondroitin sulfate, and BWA-3 that bind histones, 2G7, h2G7, glycynhizin, carbenoxolone, salicylic acid, and metformin that bind HMGB1, Hyperpolarized 1,4 fumarate that binds fumarase, and protohypericin, sennidin A, sennoside B, sennidin B, Skyrin, HQ5, IRDye800CW, HQ4, ICG, and Evans Blue (EB) that bind to not fully known markers in necroses.

In one embodiment Rhein is chemically linked through its carboxyl group to amino groups on proteins, e.g., albumin or transferrin, then the nanoparticle formed by sonicating with one or more hydrophobic drugs such as paclitaxel or other drugs listed above. This results in nanoparticles which are complexes between Rhein, protein(s), and drugs. In another embodiment Evans Blue (EB) is incubated with albumin (to which it avidly binds, or other proteins may be used) and mixed with drugs, e.g. paclitaxel or camptothecin. The mixture is then sonicated to form the treatment nanoparticles. After IV injection, the drugs are efficiently delivered to the necrotic sites since EB binds strongly to necrotic tissue. In another specific embodiment, doxorubicin (Dox), which is hydrophilic, is altered to become hydrophobic. This may be done by reacting its carboxyl group with a hydrophobic group, such as palmitic acid hydrazide, stearic acid hydrazide, or other hydrophobic hydrazides. The hydrophobic Dox is dissolved in e.g., dimethyl sulfoxide (DMSO) or PEG (e.g., 400 molecular weight), then sonicated with a carrier protein, such as albumin or transferrin to form nanoparticles. The advantages of this construct are: a) it is targeted to necrotic regions since Dox strongly binds to free DNA, plentiful in necrotic regions, b) the nanoparticle non-covalently carries a payload of additional Dox-hydrazone which will gradually be released into the region to be exposed to remaining live tumor cells, c) the Dox-hydrazone enters tumor cells and in the low pH of the endosome, the hydrazone is cleaved, releasing active Dox.

In another embodiment, doxorubicin can be combined with a protein by a disolvation or coacervation method where the protein is denatured slightly with ethanol, methanol, chloroform or gluthathione, which then binds the hydrophobic drug. Doxorubicin can be made hydrophobic by raising the pH or reacting with a hydrophobic moiety such as palmitic acid hydrazide. In a preferred embodiment we discovered that many proteins can form nanoparticles with Dox simply by mixing with the protein in water, then slowly increasing the pH with sodium hydroxide. Dox may also be encapsulated in liposomes.

A tumor treated to have vascular damage then stimulates platelet activation and fibrinogen binding as described above. This is followed by thrombin conversion of fibrinogen to fibrin which binds other fibrin molecules resulting in a fibrin thrombus. It is here disclosed to target drug, drug-protein or drug-carriers (e.g., liposomes, dendrimers, polymersomes, polymer micelles, nanospheres, silica nanoparticles, mesoporous silica nanoparticles, quantum dots, solid lipid nanoparticles, micelles, emulsions, metal nanoparticles, gold nanoparticles, magnetic nanoparticles, ferritin, carbon nanotubes, and carbon nanodiamonds) specifically to this fibrin deposit. The drug, drug-protein, or drug-carrier is targeted via substances that bind specifically to fibrin. Various peptide sequences can be used that have high specific binding to fibrin but not to fibrinogen. These include: CREKA (SEQ ID NO: 2), GPRPPGGSKGC-NH2 (SEQ ID NO: 3), GLPCDYYGTCLD (SEQ ID NO: 4), GYLCGDYTLCPD (SEQ ID NO: 5), Ac-Y(DGlu)C(HPro)YGLCYIQGK-Am (DGlu, D-glutamic acid; HPro, hydroxyproline; Ac, acetylated N terminus; Am, amidated C terminus) (SEQ ID NO: 6), KCRE (FmocLys) A (FmocLys, 9-fluorenylmethyloxycarbonyl Lys) (SEQ ID NO: 7), Ac-GNQEQVSPLTLLK (SEQ ID NO: 8), WFHCPYDLCHIL (SEQ ID NO: 9), AFHCPYDLCHIL (SEQ ID NO: 10), WAHCPYDLCHIL (SEQ ID NO: 11), WFACPYDLCHIL (SEQ ID NO: 12), WFHCAYDLCHIL (SEQ ID NO: 13), WFHCPADLCHIL (SEQ ID NO: 14), WFHCPYALCHIL (SEQ ID NO: 15), WFHCPYDACHIL (SEQ ID NO: 16), WFHCPYDLCAIL (SEQ ID NO: 17), WFHCPYDLCHAL (SEQ ID NO: 18), WFHCPYDLCHIA (SEQ ID NO: 19), QWECPYGLCWIQ (SEQ ID NO: 20), QAECPYGLCWIQ (SEQ ID NO: 21), QWECPYGLCAIQ (SEQ ID NO: 22). The sequences are best if the two internal cysteines are reduced to disulfides, thus cyclizing the peptide. Similar sequences with minor changes, especially at the amino or carboxy termini are also included, or ones that add non-specific linking sequences at either end. In a preferred embodiment, albumin is formed into a nanoparticle with Dox by the pH method disclosed (raising the pH while sonicating Dox and albumin or other protein such as transferrin) and this is linked to the cyclized fibrin-specific binding peptide Ac-Y(DGlu)C(HPro)YGLCYIQGK-Am (SEQ ID NO: 6) by standard crosslinking agents such as EDC (1-Ethyl-3-[3-dimethylaminopropyl]carbodiimide hydrochloride). The resultant nanoparticle containing a drug is then highly targeted to fibrin at the tumor site thereby delivering the drug.

The advantages of this tumor drug delivery include: a) superior targeting since there is a large quantity of easily accessible necrotic targets or fibrin deposits that are highly present at the tumor site, b) it does not interfere with platelets or clotting mechanisms and may avoid off-target toxicity, and c) superior drug delivery is achieved since the drugs are efficiently targeted.

### IX. Methods of Treating Solid Tumors

In some embodiments, provided herein is a method of treating a solid tumor comprising administering i) a treatment causing vascular damage and ii) a nanoparticle composition comprising fibrinogen and a hydrophobic chemotherapeutic agent to the individual. Without being bound by theory, in some embodiments, the treatment causing vascular damage specially damages the tumor epithelium which results in platelet activation at the site of the tumor. The nanoparticles provided herein which comprise fibrinogen are trafficked to the platelets located at the tumor. The nanoparticles then may form large aggregates in the tumor vasculature, resulting in embolization of the tumor and necrosis. Cell death at the tumor site releases proteolytic enzymes allowing release of the hydrophobic chemotherapeutic agent at the tumor site. Thus, this method allows specific targeting and release of the chemotherapeutic agent to the tumor while avoiding normal tissue.

Accordingly, in some embodiments, the methods provided herein result in activation and/or amplification of the coagulation cascade. In some embodiment, a clot or embolism is formed in the tumor vasculature. In some embodiments, the clot results in reduction of blood flow to the tumor and cell death of the tumor cells.

In some embodiments, the nanoparticles bind to activated platelets. In some embodiments, fibrinogen located on the surface of the nanoparticles bind to activated platelets. In some embodiments, each nanoparticle is able to bind to two or more platelets upon administration. In some embodiments, binding of multiple platelets by the nanoparticles results in formation of a higher order aggregate in the tumor vasculature. In some embodiments, aggregates of at least 1 μm in size are formed at the solid tumor site or the tumor vasculature. In some embodiments, aggregates of about 1 μm to about 10 μm, about 1 μm to about 5 μm, or about 3 μm to about 8 μm, or about 10 μm to about 80 μm are formed.

In some embodiments, at least about 10 mg/kg of fibrinogen is administered to the individual in the forms of the nanoparticles provided herein. In some embodiments, about 10 mg/kg to about 500 mg/kg, such as about 10 mg/kg to about 400 mg/kg, about 10 mg/kg to about 350 mg/kg, about 100 mg/kg to about 500 mg/kg, about 100 mg/kg to about 400 mg/kg, about 100 mg/kg to about 350 mg/kg, or about 200 mg/kg to about 400 mg/kg fibrinogen is administered to the individual in the form of nanoparticles comprising the fibrinogen and the chemotherapeutic agent. In some embodiments, about 350 mg/kg of fibrinogen in the form of nanoparticles is administered to the individual.

In some embodiments, the hydrophobic chemotherapeutic agent is selectively released from the nanoparticle at the site of the tumor. In some embodiments, the hydrophobic chemotherapeutic agent is not substantially released at normal tissue. In some embodiments, the hydrophobic chemotherapeutic agent is released from the nanoparticle over a period of at least about 1 hour, at least about 2 hours, at least about 3 hours, or at least about 12 hours. In some embodiments, cell lysis of tumor cells results in release of proteolytic enzymes which degrade the fibrinogen and release the chemotherapeutic agent from the nanoparticle. In some embodiments, the hydrophobic chemotherapeutic agent is released from the nanoparticle by an active release process (e.g. by protease cleavage of fibrinogen). In some embodiments, the hydrophobic chemotherapeutic agent is released from the nanoparticle by a passive process (e.g. diffusion).

In some embodiments the treatment causes selective damage of the solid tumor. In some embodiments, surrounding tissue is not significantly damaged. In some embodiments, normal tissue is not significantly damaged.

In some embodiments, the treatment provided herein triggers an immune response at the site of the solid tumor. In some embodiments, immune cells, such as cytotoxic T cells are stimulated upon administration of the treatments provided herein. In some embodiments, pro inflammatory cytokines are released following treatment. In some embodiments, tumor lysis caused by administration of the compositions provided herein results in an immune response.

In some embodiments, the method comprises administering i) a treatment causing vascular damage and ii) a nanoparticle composition comprising fibrinogen and a hydrophobic chemotherapeutic agent to the individual, wherein the hydrophobic chemotherapeutic agent is paclitaxel. In some embodiments, the paclitaxel is administered at a dose of about 10 mg/kg to about 100 mg/kg, such as about 30 mg/kg to about 90 mg/kg, about 30 mg/kg to about 80 mg/kg, about 30 mg/kg to about 70 mg/kg, about 30 mg/kg to about 60 mg/kg, about 50 mg/kg to about 90 mg/kg, about 60 mg/kg to about 90 kg, or about 70 mg/kg to about 90 mg/kg in the form of nanoparticles provided herein. In some embodiments, the paclitaxel chemotherapeutic agent, or fibrinogen dose is determined based upon the amount present in the nanoparticles delivered to the individual.

In some embodiments, the treatment causing vascular damage is administered intravenously. In some embodiments, the treatment causing vascular damage is administered intratumorally. In some embodiments, the treatment causing vascular damage is administered intraperitoneally. In some embodiments, the vascular disrupting agent is administered intravenously. In some embodiments, the vascular disrupting agent is administered intratumorally. In some embodiments, the vascular disrupting agent is administered intraperitoneally.

In some embodiments, the composition comprising the nanoparticles is administered intravenously. In some embodiments, the composition comprising the nanoparticles is administered intratumorally. In some embodiments, the composition comprising the nanoparticles is administered intraperitoneally.

The composition comprising the nanoparticles and the treatment causing vascular damage can be administered simultaneously or sequentially, in any order. In some embodiments, the composition comprising the nanoparticles is administered prior to the treatment causing vascular damage. For example, in some embodiments the composition comprising the nanoparticles is administered at least about 5 minutes, at least about 10 minutes, at least about 20 minutes, or at least about 60 minutes, or at least 4 hours before the treatment causing vascular damage.

In some embodiments, the treatment causing vascular damage is administered prior to the composition comprising the nanoparticles. In some embodiments the treatment causing vascular damage is administered at least about 10 minutes, at least about 20 minutes, or at least about 60 minutes, or at least 4 hours before the composition comprising the nanoparticles. In some embodiments, the composition comprising the nanoparticles are administered simultaneously, such as within 10 minutes, within 5 minutes, or within 3 minutes of one another.

The methods provided herein are useful for treating a wide range of tumors due to their general mechanism of action. In some embodiments, the solid tumor is selected from the group consisting of lung and bronchus, breast, prostate, colon, rectal, melanoma, bladder, kidney, endometrial, pancreatic, thyroid, liver, intrahepatic bile duct, gastrointestinal, brain and nervous system, cervical, head and neck, ovarian, testicular, eye, skin, lymphomas and bone and muscle sarcomas.

### X. Kits

Also provided herein are kits comprising i) a composition comprising nanoparticles comprising a hydrophobic chemotherapeutic agent and fibrinogen and ii) a vascular disrupting agent. In some embodiments, the hydrophobic chemotherapeutic agent is selected from the group consisting of paclitaxel, camptothecin, docetaxel, and artemisinin. In some embodiments, the vascular disrupting agent is selected from the group consisting of DMXAA, CA4P, Plinabulin, CKD-516, AVE8062, AVE9062 OXi4503, MPC6827, BNC105P, ABT-751, VEGF-gelonin, Verubulin, and flavone-8-acetic acid (FAA). In some embodiments, the kit provides instructions for use according to any of the methods provided herein.

### XI. Methods of Making Nanoparticles

Also provided herein are methods of making nanoparticles and/or compositions comprising nanoparticles. In one embodiment, hydrophobic molecules or substances are sequestered by proteins. This is proposed to be achieved since proteins have many hydrophobic residues and under certain conditions these may bind to the hydrophobic payload. In particular, almost any protein can be employed, but in some embodiments the proteins are chosen from the group fibrinogen, transferrin, albumin, antibodies, and antibody fragments. The proteins can be used separately or in combination. The complex formation was found to be in some embodiments attained by sonication of the payload and protein. For some hydrophobic drugs, they are first dissolved in a solvent which can be polyethylene glycol, methoxy polyethylene glycol, dimethoxy polyethylene glycol, all of various molecular weights, but also including the range of 100-1,000. Another useful solvent for some materials was found to be dimethysulfoxide. Other solvents include ethanol, methanol, dimethylformamide, acetone, acetonitrile, alcohols, dioxane, tetrahydrofuran, chloroform, toluene, ethyl acetate, cyclohexane, diethy ether, and hexane. The dissolved hydrophobic payload is then sonicated with the protein to form the complex. In other embodiments sonication can be replaced by high pressure homogenization, homogenization, mechanical mixing, ball milling, or solvent evaporation. The mixing can be done in a temperature-controlled water or ice water bath to control protein denaturation. The dried forms of both protein and payload (e.g., drug), or having one component in a solvent, can be complexed by grinding with a mortar and pestle, dounce, or similar equipment.

In some embodiments, the method comprises sonicating fibrinogen and a hydrophobic chemotherapeutic agent. In some embodiments, the method comprises dissolving fibrinogen in a buffered solution and heating the dissolved fibrinogen at 37° C. for at least 5 minutes.

### Exemplary Embodiments

The present disclosure may be better understood with reference to the following exemplary embodiments.

Embodiment 1. A treatment process with the steps of a) creating or using vascular damage, producing activated platelets, and b) targeting the activated platelets with nanoparticles.

Embodiment 2. The process of claim 1 where vascular damage is created by vascular disrupting agents, radiation, or heat.

Embodiment 3. The nanoparticles of claim 1 that contain fibrinogen or other molecules that bind to activated platelets.

Embodiment 4. The nanoparticles of claim 1 that contain paclitaxel, camptothecin, docetaxel, artemisinin, or other drugs.

Embodiment 5. The nanoparticles of claim 1 that contain transferrin or albumin.

Embodiment 6. The process of claim 1 used to treat cancers.

Embodiment 7. A treatment process with the steps of a) creating or using necrotic regions, and b) targeting the necrotic regions with nanoparticles.

Embodiment 8. The treatment process of claim 7 where the necrotic regions are produced by the process of claim 1.

Embodiment 9. The nanoparticles of claim 7 that contain molecules that bind necrotic regions or free DNA including acridines, acridine orange, acridine yellow G, anthracyclines, and doxorubicin.

Embodiment 10. The nanoparticles of claim 7 that contain paclitaxel, camptothecin, docetaxel, artemisinin, or other drugs.

### Examples

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. The examples below are intended to be purely exemplary of the application and should therefore not be considered to limit the application in any way.

All publications and patent applications mentioned in this specification are herein incorporated by reference to the same extent as if each individual publication or patent application was specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### Example 1: Synthesis of Fibrinogen-Paclitaxel Nanoparticle

12 mg of fibrinogen was dissolved in 66 ul of 1 molar Tris pH 8.0 plus 33 ul of water. Once dissolved, 200 ul of phosphate buffered saline, pH 7.4 (PBS) was added. Paclitaxel (2 mg) was dissolved in 75 ul of 400 molecular weight (MW) polyethylene glycol (PEG). It was heated to 80° C. for 2 minutes, vortexed then heated again to 80° C. for 2 minutes. The two solutions were combined and sonicated in an ice-water bath for 2 minutes. 720 ul of PBS was added and the solution sonicated for an additional 6 minutes. It was then filtered through a 1 μm filter.

### Example 2: Cancer Treatment

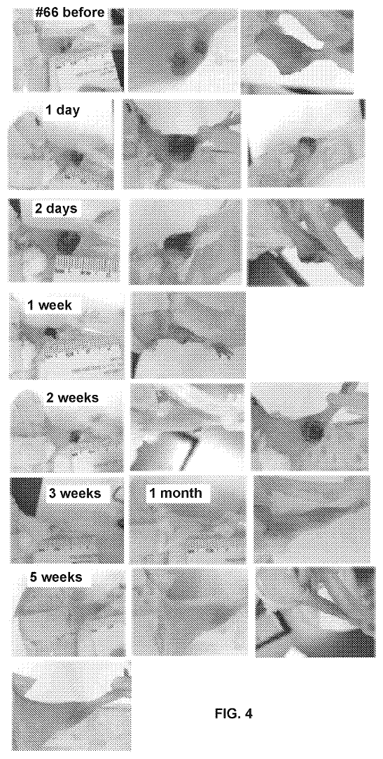

EMT-6 mouse mammary tumors were grown in syngeneic Balb/C mice by subcutaneously injecting tumor cells into the leg. When grown to an appropriate size, 100-500 mm³, mice were intravenously injected in the tail vein with 60 mg PTX/kg of the nanoparticle solution of Example 1. Two minutes later a vascular disrupting agent (25 mg/kg of DMXAA dissolved in 100 mM Tris pH 8 buffer) was administered intraperitoneally. The tumors became dark colored and then black after one day indicating necrosis, and progressed on to shrink and lose material becoming a scab that eventually disappeared. Examples of treated tumor bearing mice are shown in FIGS. 4-7.

### Example 3: Synthesis of Fibrinogen-Camptothecin Nanoparticle

12 mg of fibrinogen was dissolved in 66 ul of 1 molar Tris pH 8.0 plus 33 ul of water. Once dissolved, 200 ul of phosphate buffered saline, pH 7.4 (PBS) was added. Camptothecin (0.2 mg) was dissolved in 40 ul DMSO. It was heated to 80° C. for 2 minutes, vortexed then heated again to 80° C. for 2 minutes. The two solutions were combined, then sonicated in an ice-water bath for 2 minutes. 720 ul of PBS was added and the solution was sonicated for an additional 6 minutes. It was then filtered through a 1 μm filter.

### Example 4: Synthesis of Fibrinogen (Fg)-Transferrin (Tf)-Camptothecin (CPT) Nanoparticles

12.5 mg Fg was dissolved in 66 ul of 1 molar Tris pH 8.0 plus 33 ul of water. 5.6 mg Tf was dissolved in 200 μl of PBS. 0.2 mg CPT was dissolved in 50 ul of DMSO. The protein solutions were combined and 0.7 mL of PBS was added. While this solution was being sonicated, the CPT solution was slowly injected at the sonicator tip over 1 min at room temperature. The solution was sonicated 3 min more in an ice bath and filtered through a 1 μm nylon filter.

### Example 5: Cancer Treatment

The nanoparticles of Example 4 (5.4 mg CPT/kg) were intravenously injected into a mouse bearing an EMT-6 subcutaneous breast tumor. Two min later, 22 mg DMXAA/kg was injected intraperitoneally. The following day the tumor became very dark and on the second day became black indicating extensive necrosis.

### Example 6: Synthesis of Fibrinogen-Transferrin-Docetaxel (DTX) Nanoparticles

5.5 mg fibrinogen was dissolved in 66 ul of 1 molar Tris pH 8.0 plus 33 ul of water. 5 mg transferrin was dissolved in 200 μl of PBS. 2 mg of docetaxel was dissolved in 75 ul of PEG 400 mw. To completely dissolve the docetaxel it was heated to 80° C. for 2 minutes in a dry bath (heater block), vortexed, then repeated 2 or 3 times until fully dissolved. The docetaxel was added to the transferrin solution and sonicated with a tip sonicator inserted into the solution for 2 min on ice. 720 ul of PBS was added and it was further sonicated for 3 min. on ice. The fibrinogen solution was then added and sonicated for an additional 6 min. on ice. It was filtered with a 1 μm filter before intravenous injection.

### Example 7: Synthesis of Fibrinogen-Docetaxel Nanoparticles

10 mg fibrinogen was dissolved in 200 ul 1M Tris pH 8.0 and 100 ul of water. 2 mg DTX as dissolved in 75 ul PEG 400 MW, heated to 80° C. for 2 min, vortexed, and reheated/vortexed 2 additional times. The two solutions were mixed and sonicated 2 min in an ice bath. 0.4 mL water was added and sonicated 3 min. 0.6 mL water was added and sonicated (all in an ice bath) for 3 min. The product was filtered with a 5 μm filter before intravenous injection.

### Example 8: Synthesis of Transferrin (Tf)-Paclitaxel (PTX) Nanoparticles

12 mg Tf was dissolved in 400 μl of PBS. 1.1 mg of PTX was dissolved in 100 ul of PEG 400 and heated at 80° C. for 2 min, vortexed (repeated twice). The two solutions were mixed and sonicated in an ice bath for 11 min. The product was filtered with a 0.45 μm filter before intravenous injection.

### Example 9: Cancer Treatment with Tf-PTX Nanoparticles

EMT-6 mouse mammary tumors were grown in syngeneic Balb/C mice by subcutaneously injecting tumor cells into the leg. When tumors reached ˜500 mm³, the Tf-PTX nanoparticles of Example 8 were intravenously injected at 28 mg PTX/kg weekly. This treatment appeared to stop further growth of the tumors.

### Example 10: Synthesis of Artemisinin (Art)-Fibrinogen (Fg)-Transferrin (Tf) Nanoparticles

4.5 mg Fg was dissolved in 33 ul 1M Tris pH 8.0 and 66 ul water. 5.6 mg Tf was dissolved in 200 ul PBS. The two protein solutions were mixed with addition of 0.7 mL of PBS. 2 mg Art was dissolved in 50 ul dimethysulfoxide (DMSO). The protein solution was sonicated and the Art solution was slowly added at the sonicator tip. The sample was filtered with a 1 μm nylon filter.

### Example 11: Cancer Treatment Using Artemisinin (Art)-Fibrinogen (Fg)-Transferrin (Tf) Nanoparticles

EMT-6 mouse mammary tumors were grown in syngeneic Balb/C mice by subcutaneously injecting tumor cells into the leg. When grown to an appropriate size, 100-500 mm³, mice were intravenously injected in the tail vein with the Art-Fg-Tf nanoparticles of Example 10 administering 50 mg Art/kg. 10 minutes later 25 mg DMXAA/kg of vascular disrupting agent was given intraperitoneally. The following day the tumors became black, indicating extensive necrosis.

### Example 12: Synthesis of Nanoparticles Containing Multiple Drugs (Fg-PTX-CPT)

12 mg of fibrinogen was dissolved in 66 ul of 1 molar Tris pH 8.0 plus 33 ul of water. Once dissolved, 200 ul of phosphate buffered saline, pH 7.4 (PBS) was added. Paclitaxel (2 mg) was dissolved in 75 ul of 400 molecular weight (MW) polyethylene glycol (PEG). It was heated to 80° C. for 2 minutes, vortexed then heated again to 80° C. for 2 minutes. 0.2 mg CPT was dissolved in 50 ul of DMSO. The three solutions were combined and sonicated in an ice-water bath for 2 minutes. 720 ul of PBS was added and the solution sonicated for an additional 6 minutes. It was then filtered through a 1 μm filter.

### Example 13: Nanoparticles Targeting DNA

0.2 mg of acridine yellow G or 0.2 mg of propidium iodide were dissolved in DMSO by heating twice at 80° C. for 2 min. 15 mg albumin was dissolved in 0.4 mL of PBS. While sonicating the albumin solution in an ice bath, the acridine or propidium solutions were slowly over about 10-20 sec injected at the sonicator tip. Sonication was continued for another minute. Clear colored solutions were obtained. Both were filtered through a 0.45 μm filter, all passing through. However, if albumin was not used, the solution containing acridine all was retained by the filter, whereas the propidium solution passed through. Both were purified with 50 kD Amicon centrifugal filters with PBS washing. It was found that the acridine yellow remained in the retentate, whereas the propidium came through in the filtrate, indicating that a stable complex with the albumin was only attained with the acridine yellow G. The purified albumin-acridine yellow G was applied to about a 1 mg clump of DNA and exposure to a UV lamp indicated high uptake (the DNA strands had high fluorescense). Fluoresence microscopy also revealed the DNA had become fluorescent, even after washing thoroughly with PBS. These results indicated that the albumin-acridine complexes targeted to and bound DNA.

### Example 14: Drug-Nanoparticles Targeting DNA Using Doxorubicin (Dox)

20 mg of albumin was dissolved in 0.4 mL water and mixed with 0.5 mg of Dox making a clear colored solution. Under sonication and in an ice-water bath, 80 ul of 0.1 M NaOH was slowly injected near the sonicator tip. Next, 10 ul of 8% glutaraldehyde (in water) was added with an additional 5 minutes of sonication producing a clear solution. After 30 min, 30 ul of a 3 M solution of ethanolamine hydrochloride was added. After 30 min, the construct was purified on a 50 kDa centrifugal filter. None of the Dox color came through the filter indicating it was bound to the albumin. The retentate was resuspended in phosphate buffered saline (PBS) and re-centrifuged on the 50 kDa filter. After resuspension of the retentate in PBS the size of the nanoparticles was measured to be 117 nm by dynamic light scattering. The product was mixed with strands of DNA and after 1 hour the color (from the Dox) appeared only bound to the DNA showing that the nanoparticle was targeted to DNA.

### Example 15: Drug-Nanoparticles Targeting Fibrin

12 mg of albumin or transferrin is dissolved in 300 μl of PBS and mixed with 3.6 mg paclitaxel (PTX) that is dissolved in 75 ul of 400 molecular weight polyethylene glycol (PEG 400) and sonicated for 3 min. An additional 660 ul of PBS is added and sonicated for 6 minutes yielding protein-drug nanoparticles of about 130 nm in diameter. Next, these are reacted with 50 mg of DMTMM (4-(4,6-dimethoxy-1,3,5-triazin-2-yl)-4-methyl-morpholinium chloride) for 20 minutes at 50° C. The nanoparticles are then purified from excess DMTMM by 3 washes using 50 kDa centrifugal filters. The particles are then incubated for 16 hours at 37° C. with the fibrin-targeting peptide Ac-Y(DGlu)C(HPro)YGLCYIQGK-Am (SEQ ID NO: 6), where DGlu=D-glutamic acid; HPro=hydroxyproline; Ac=acetylated N terminus; and Am=amidated C terminus. The product is purified from excess peptide using 3 washes with PBS using 50 kDa centrifugal filters. The final product is then intravenously injected into mice having tumor fibrin deposits at 90 mg PTX/kg. This results in extraordinary drug delivery and tumor treatment efficacy.

### Example 16

Fibrinogen-paclitaxel nanoparticles were constructed essentially as detailed in Example 1 except amounts were adjusted to the animal weight to intravenously inject 90 mg/kg (body weight) of paclitaxel and 350 mg/kg fibrinogen. Two minutes after, 18 mg/kg DMXAA was injected intraperitoneally. Before treatment the advanced subcutaneous syngeneic EMT6 mouse mammary grown in a Balb/C mouse had a volume of approximately 900 mm³. One day after treatment the tumor became black in color and mostly necrotic. This progressed over one week with the tumor shrinking to about one-fourth of its original volume. After 1 month, no tumor was detectable, leaving only a slight skin discoloration. The results are shown in FIG. 4. Animals treated this way show complete eradication of tumors with no recurrence, so far to 9 months. Lifespan of a mouse is typically 18 months.

### Example 17

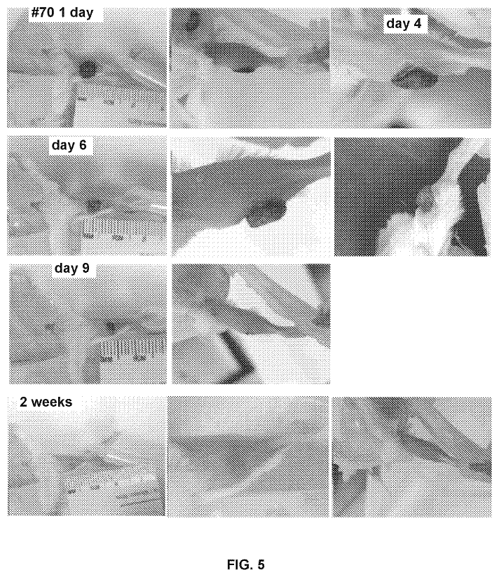

Fibrinogen-paclitaxel nanoparticles were constructed essentially as detailed in Example 1 except amounts were adjusted to the animal weight to intravenously inject 60 mg/kg (body weight) of paclitaxel and 350 mg/kg fibrinogen. Two minutes after, 18 mg/kg DMXAA was injected intraperitoneally. Before treatment the subcutaneous syngeneic EMT6 mouse mammary grown in a Balb/C mouse had a volume of approximately 100 mm³. One day after treatment the tumor became black in color and mostly necrotic. This progressed over one week with the tumor shrinking. After 2 weeks, no tumor was detectable, leaving only a slight skin discoloration. The results are shown in FIG. 5.

### Example 18

Fibrinogen-paclitaxel nanoparticles were constructed essentially as detailed in Example 1 except amounts were adjusted to the animal weight to intravenously inject 59 mg/kg (body weight) of paclitaxel and 232 mg/kg fibrinogen. Two minutes after, 25.8 mg/kg DMXAA was injected intraperitoneally. Before treatment the advanced subcutaneous syngeneic EMT6 mouse mammary grown in a Balb/C mouse had a volume of approximately 900 mm³. One day after treatment the tumor became black in color and mostly necrotic on the second day. Tumor shrinkage continued and by 1½ weeks it was less than half of its starting size. The results are shown in FIG. 6.

### Example 19

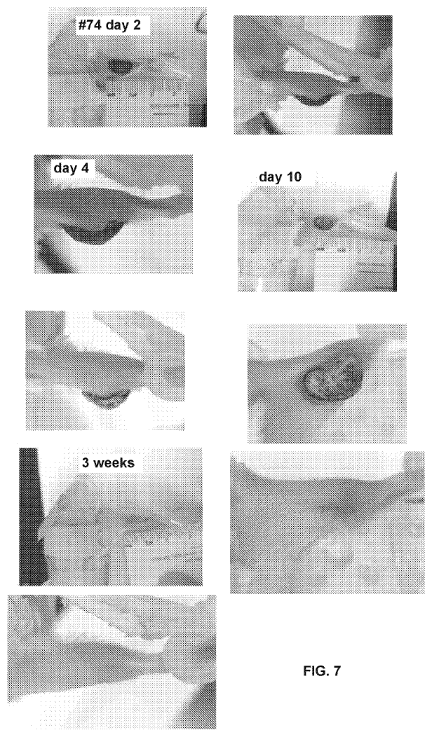

Fibrinogen-paclitaxel nanoparticles were constructed essentially as detailed in Example 1 except amounts were adjusted to the animal weight to intravenously inject 90 mg/kg (body weight) of paclitaxel and 350 mg/kg fibrinogen. Two minutes after, 18 mg/kg DMXAA was injected intraperitoneally. Before treatment the advanced subcutaneous syngeneic EMT6 mouse mammary grown in a Balb/C mouse had a volume of approximately 800 mm³. One day after treatment the tumor became black in color and by day 2 mostly necrotic. The tumor mass continued to shrink, becoming a dead scab. This later fell off and no tumor was detected 3 weeks after treatment. The results are shown in FIG. 7.

### Example 20

A Clauss clotting assay was used to compare the clotting activity of fibrinogen with fibrinogen nanoparticles. The fibrinogen samples at various concentrations were mixed with thrombin and the clotting observed over time in a spectrophotometer at 405 nm.

### Fibrinogen (Fgn) Nanoparticle Preparation

14 mg of Fgn (bovine, J63276 from ThermoFisher, CAS #9001-32-5) was dissolved in a solution of 900 μL PBS and 100 μL 0.5 M Borate buffer. 2.33 mg of paclitaxel was dissolved in 50 μL of polyethylene (PEG) 400 molecular weight (MW) and heated at 80° C. until completely dissolved with occasional vortexing. The PEG solution was added to the Fgn solution and sonicated with a tip sonicator (Misonix Microson XL2000 Ultrasonic Cell Distruptor with Micro Probe Tip) at 4.5 power setting in an ice water bath until the solution turned clear (about ten minutes). 1 mL 0.1M Tris Buffered Saline (TBS) was added to the 1 mL Fgn NP solution, bringing the Fgn NP solution to a final concentration of 7 mg/mL.

### Assay

1, 2, and 4 mg/mL Fgn alone and Fgn NPs were prepared in TBS, 500 μL final volume. 500 μL of the solution was added to a quartz cuvette and blanked in a spectrophotometer. 5 μL of a thrombin solution (0.104 NIH units, MP Biochemicals) was added to the cuvette and the 500 μL solution was pipetted up and down twice with a 1 mL manual pipette, set to 500 μL volume. Then the absorbance at 405 nm was measured continuously in overlay mode over 4 minutes until 30 spectra were recorded. The absorbance at 405 was then graphed. This was done for 1, 2, and 4 mg/mL of Fgn alone and Fgn nanoparticles. Also 4 mg/mL of Fgn alone and Fgn NPs alone were recorded without thrombin addition as controls.

### Results

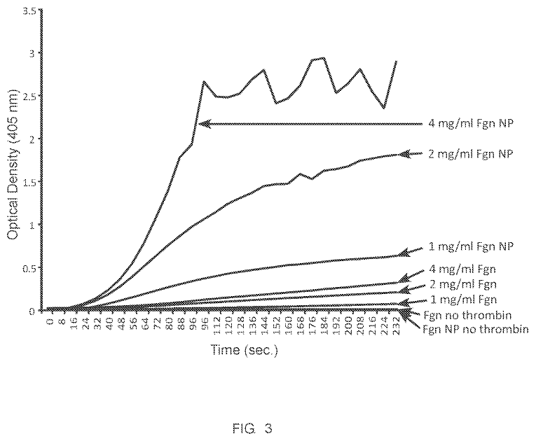

The nanoparticles clotted at a much higher rate than fibrinogen by itself (FIG. 3). The normal average blood concentration is about 2 mg/ml and this is compared to a treatment fibrinogen nanoparticle concentration of 6 mg/mL (concentration in blood after intravenous injection, 240 mg/kg). At these comparative levels (2 mg/ml Fgn compared to 6 mg/ml Fgn nanoparticles), the fibrinogen nanoparticles caused coagulation at a rate 30 times that of endogenous fibrinogen. Fgn and Fgn nanoparticles without thrombin showed no clotting activity.

### Example 21: Fibrinogen Nanoparticles Formed Using Denaturants

These may be formed without anti-cancer drugs, and are useful to enhance clotting or target vascular damage for imaging or drug delivery.

### Example 22: Fibrinogen Nanoparticles Formed Using Hydrophobic Substances

These may be formed without anti-cancer drugs, and are useful to enhance clotting or target vascular damage for imaging or drug delivery.

### Example 23: Transferrin (Tf)-Drug Nanoparticles and Transferrin-Fibrinogen-Drug Nanoparticles

Transferrin receptors are found upregulated in tumors due to their higher metabolism and need for iron. 14 mg of Tf or 7 mg of Tf and 7 mg fgn were dissolved in a solution of 900 μL PBS and 100 μL 0.5 M Borate buffer. 2.33 mg of paclitaxel was dissolved in 50 μL of polyethylene (PEG) 400 molecular weight (MW) and heated at 80° C. until completely dissolved with occasional vortexing. The PEG solution was added to the Fgn solution and sonicated with a tip sonicator in an ice water bath until the solution turned clear (a few minutes). 1 mL 0.1M Tris Buffered Saline (TBS) was added to the 1 mL NP solution, bringing the NP solution to a final concentration of 7 mg/mL.

### Example 24: Plasminogen-Depleted Fibrinogen Nanoparticle Preparation

The fibrinogen solution was first made by adding 7 microliters Tris pH 8 buffer and 4 microliters DI (deionized) water and 90 microliters PBS to 1 mg plasminogen-depleted-fibrinogen. The paclitaxel solution was made by dissolving 0.2 mg paclitaxel in 10 microliters ethanol. The fibrinogen solution was added to paclitaxel solution and mixed. 2 microliters of glutaraldehyde (8% in water) was added and sonicated for 3 minutes. Ethanolamine (4 mg in 20 μL PBS) was added to stop the reaction and sonicated for 3 minutes. The size measured by dynamic light scattering was <200 nm. The nanoparticles were purified by centrifuging them at 21 Kg for 5 minutes and the supernatant transferred to a new tube. Saturated ammonium sulfate solution was added to a final concentration of ammonium sulfate 33%. The sample was centrifuged at 21 Kg for 5 minutes. The supernatant was discarded and the pellet resuspended in 200 microliters PBS. This was sonicated for 2 minutes and the size again was <200 nm.

### Example 25: Modified Fibrinogen-Paclitaxel Preparation for Nanoparticles ˜122 nm with Low Polydispersity (<0.15)

One ml of buffer was prepared using 950 μL of PBS and 50 μL of 0.5 M Borate Buffer, pH 8.5. 14 mg of fibrinogen (75% clottable) was dissolved in the 1 mL buffer and vortexed so the Fgn fully dissolved and the solution was clear. 2.33 mg of paclitaxel was dissolved in 50 μL PEG 400 and heated at 80° C. for ten minutes with occasional vortexing. The PTX solution was completely clear. The solution was cooled for one minute and added to the bottom of the 1 mL Fgn solution. The bottom layer was turbid where the PEG-drug was added, and the top of the solution stayed clear. It was preferable to add the PEG-drug to the Fgn and not the Fgn to the PEG-drug. The sample was sonicated for ten minutes in a water bath that had been cooled with ice previously, and was cold from the melted ice, but had no additional ice that was added during sonication. The solution turned almost completely clear after 3 minutes and stayed almost clear for the rest of the sonication. DLS (dynamic light scattering) measured nanoparticles of 122 nm with a polydispersity of 0.123.

### Example 26: PEGylation of Nanoparticles

The nanoparticles can be pegylated to increase stability and extend blood half-life. 100 microliters 0.5M 1 kD amino terminated PEG, pH 6.4 was added to fgn-drug nanoparticle solution and sonicated while adding 2 μL of glutaraldehyde (8% in water). Sonication was continued for 5 minutes. The sample was centrifuged at 21 Kg for 3 minutes. The supernatant was discarded and the pellet resuspended in 1 mL PBS.

### Example 27: Freeze Drying of Nanoparticles

The fgn-paclitaxel nanoparticles were freeze dried to a powder and after 1 week of storage at room temperature were resuspended in water to their original volume and found to have the same size and polydispersity as the original sample. This indicates the nanoparticles can be preserved after preparation for useful shelf-life.

### Example 28: Fgn-Oil Red O Nanoparticles and Albumin-Oil Red O Nanoparticles

13.8 mg of fgn and albumin were separately dissolved in 950 μL PBS and 50 μL Borate buffer, pH 8.5. 1.186 mg of Oil Red O was dissolved in 75 μL of PEG 400. The Oil Red O/PEG solution was heated for 10 minutes and vortexed until completely dissolved. The Oil Red O/PEG solution was pipetted into the fgn or albumin solutions and sonicated for 10 minutes. Fgn-Oil Red O nanoparticles were formed with a size of 170 nm and polydispersity of 0.18. Nanoparticles were then precipitated with ammonium sulfate and spun down. The supernatant was completely clear and the nanoparticle pellet was red in color, indicating that all of the Oil Red O was incorporated within the nanoparticles. The nanoparticles were next precipitated with ethanol and sonicated, followed by centrifugation. The pellet was white in color and the supernatant was completely red, indicating the Oil Red O was released from the nanoparticles after ethanol and sonication.

### Example 29: Targeting Ear Vascular Damage In Vivo with Fibrinogen-Oil Red O Nanoparticles Compared to Albumin-Oil Red O Nanoparticles

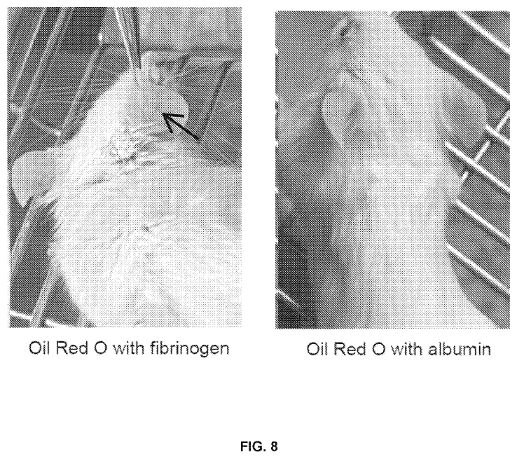

The fgn or albumin oil red o nanoparticles were intravenously injected in separate mice followed by pinching of the right ears to create mild vascular damage. Two minutes later, the fgn-oil red o clearly showed vascular accumulation by the red color only in the right car (arrow in FIG. 8). The albumin-oil red o did not show any red accumulation (FIG. 8).

### Example 30: Doxorubicin (Dox)-Protein Nanoparticles

In a 13 mL test tube, 50 microliters PEG 400, 7 mg Doxorubicin, 7 mg Palmitic acid hydrazide and 12 mg DMTMM were dissolved in 8 mL low-water methanol. For complete dissolution, sonication was applied for a few minutes. The solution was transferred to a 100 mL pear shaped flask and rotary-evaporated to dryness in a 60 degrees Celsius water bath. The solid was dissolved in 1 mL methanol and silica added, again being rotary evaporated till dryness. The contents were loaded onto a silica column and eluted with 10% and 20% methanol in dichloromethane. The solvent of the Dox peak was evaporated, and the hydrophobic Dox dissolved in 50 microliters of DMSO. 12 mg of either fgn or albumin were dissolved in 0.9 mL of PBS and mixed with the DMSO-Dox solution and sonicated for 4 minutes. The size of the resultant nanoparticles was approximately 150 nm.

### Example 31: a Preferred Synthesis of Fibrinogen-Paclitaxel Nanoparticles

Lyophilized Fibrinogen (fgn) was human fgn (FIB 1, plasminogen-depleted) from Enzyme Research Laboratories, Inc. The preparation was scaled to make 1 mL which was then filtered and 0.5 mL injected into a 25.5g mouse. The injected amounts were 350 mg/kg fibrinogen and 88 mg/kg paclitaxel. A) 17.8 mg of FGN was dissolved in 25 ul 500 mM Borate, 50 uL 3M NaCl, and 780 ul deionized (DI) water, added sequentially onto the solid Fgn, without mixing. The solution was heated at 37° C. for 15 minutes. After 15 minutes, the solution was pipetted up and down with a manual pipette to ensure complete mixing. B) 4.5 mg of PTX was mixed with 30 ul of PEG 200 MW. This was heated for ten minutes at 80° C. to fully dissolve. C) The PEG-PTX solution was removed from heat and quickly added to the bottom of the FGN solution. The solution was sonicated for 3 minutes at 4.5 power with a tip sonicator (55 watt Qsonica.Q55, ⅛″ probe). D) 125 ul of 10× Phosphate Buffer pH 7.4 (no NaCl) was then added to the solution and vortexed. The solution was sonicated for 3 minutes at 4.5 power. E) Dynamic Light Scattering (DLS) of the solution showed the size to be, 282 nm with 0.242 polydispersity (PD). The solution was filtered through a 1-micron nylon syringe filter and re-measurement showed 244 nm, 0.174 PD. F) 0.5 mL was intravenously injected into a 25.5g mouse. G) Immediately after, 18 mg/kg of the vascular disrupting agent DMXAA was given intraperitoneally. The DMXAA solution was prepared by dissolving 1 mg in 40 μL of 1M Tris pH 8 buffer, then adding 360 μL of water for a final concentration of 2.5 mg/mL.

### Example 32: Production of Nanoparticles that do not Aggregate in Serum

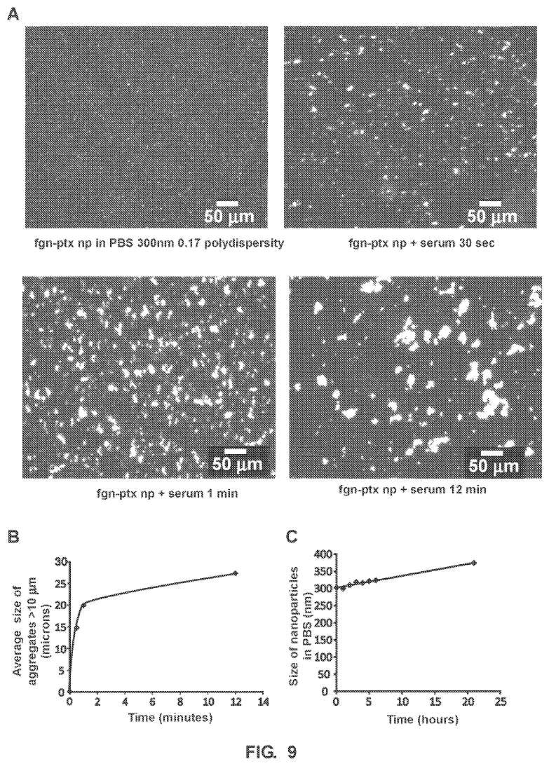

In some cases, nanoparticles bind to and crosslink with serum proteins when mixed with serum. As shown in FIGS. 9A and 9B, the nanoparticles are passivated so that they do not interact with serum proteins and form aggregates when mixed with serum. Fibrinogen-paclitaxel nanoparticles were constructed as described in Example 31. 200 μL was placed in an Eppendorf tube and the sonicator tip placed close to the bottom. 50 μL of a 200 mg/ml solution of albumin was slowly injected at the tip while sonicating over a period of 1 minute using a syringe, then sonicated further for 3 minutes. The nanoparticle by itself was relatively stable in physiological solution (phosphate buffered saline, pH 7.4), not changing in size over one hour (FIG. 9C). The size of the nanoparticle in PBS was found to be 303 nm with a polydispersity of 0.28 when measured by dynamic light scattering. When mixed with fetal calf serum, aggregates rapidly form, even at 30 seconds (FIGS. 9A and 9B). When I part nanoparticle solution at time 0 was mixed with 3 parts fetal calf serum, the size was found to be stable and did not increase. A day later when mixed with PBS or serum, the nanoparticle size was 343 nm with a polydispersity of 0.23. Two days later when mixed with serum, the size was 338 nm with a polydispersity of 0.30. Therefore, nanoparticles sizes were stable despite aggregation in fetal calf serum. This aggregation property of the nanoparticles was also found to be blocked by properly applying serum or albumin in excess during particle preparation, in essence providing a protective corona that does then not induce aggregation of the stable nanoparticles when further exposed to serum.

When the nanoparticles that aggregate in serum were intravenously given to mice without tumors, no obvious toxicity was observed and weight gain was normal. The aggregates are non-covalently linked, apparently allowing passage through the vascular system. However, if the animal was bearing a tumor and was primed with the VDA DMXAA, the aggregates accelerated the blockage of blood vessels since the fibrinogen avidly bound these larger structures to activated platelets, causing crosslinking of the aggregates and more effective embolization.

### Example 33: The Treatment Process

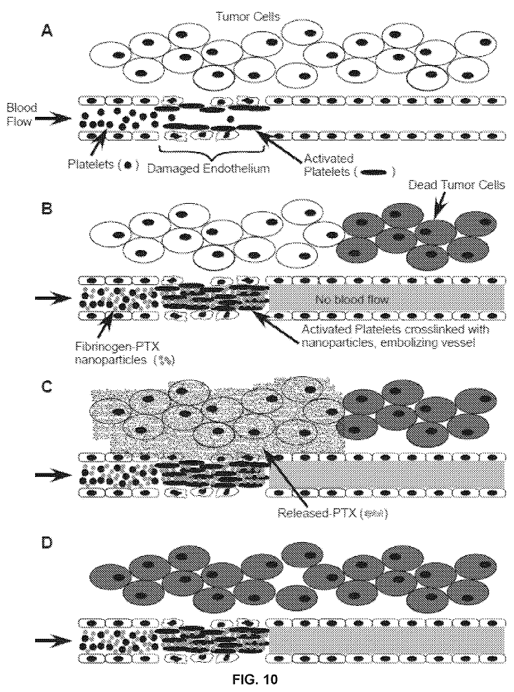

The first step of the treatment process is to damage tumor endothelium using vascular disrupting agents, radiation, heat, ultrasound, or other mechanical or chemical means (FIG. 10A). Damaged endothelium exposes subendothelial components, such as tissue factor and collagen, that lead to binding of circulating platelets and their activation. The platelets dramatically change their shape from discoid to spread-out forms, which results in greater surface area and change in their surface properties. Activated platelets expose the active integrin GPIIb/IIIa on their surface, which avidly binds circulating fibrinogen (FIG. 10B). Fibrinogen-drug (e.g., paclitaxel, PTX) nanoparticles amplify this aggregation with platelets resulting in occlusion of tumor vessels. Tumor cells downstream are cut off from oxygen and nutrients, resulting in tumor cell death. However, tumor cells at the tumor's periphery obtain oxygen and nutrients from nearby normal vasculature and survive. The accumulated Fgn-PTX nanoparticles in the thrombis break down and release the drug contents, which diffuses to nearby tumor cells that may have survived (FIG. 10C). Additionally, the dead tumor cells release proteolytic enzymes that accelerate breakdown of the fibrinogen protein, further releasing the drug. The treatment process ultimately results in effective and thorough tumor cell death (FIG. 10D).

### REFERENCES CITED

Adams, G. P.; Schier, R.; McCall, A. M.; Simmons, H. H.; Horak, E. M.; Alpaugh, R. K.; Marks, J. D.; Weiner, L. M. High affinity restricts the localization and tumor penetration of single-chain fv antibody molecules. Cancer Res 2001, 61, 4750-4755.
Andrew S M, Johnstone R W, Russell S M, Mckenzie I F, Pietersz G A. Comparison of in vitro cell binding characteristics of four monoclonal antibodies and their individual tumor localization properties in mice. Cancer Res. 1990 Jul. 15; 50 (14): 4423-8.
Bhattacharyya J, Joseph J Bellucci, Isaac Weitzhandler, Jonathan R McDaniel, Ivan Spasojevic, Xinghai Li, Chao-Chieh Lin, Jen-Tsan Ashley Chi, Ashutosh Chilkoti. Nat Commun. 2015 Aug. 4:6:7939. doi: 10.1038/ncomms8939. A paclitaxel-loaded recombinant polypeptide nanoparticle outperforms Abraxane in multiple murine cancer models
Banerjee, I.; De, K.; Mukherjee, D.; Dey, G.; Chattopadhyay, S.; Mukherjee, M.; Mandal, M.; Bandyopadhyay, A. K.; Gupta, A.; Ganguly, S.; et al. Paclitaxel-loaded solid lipid nanoparticles modified with Tyr-3-octreotide for enhanced anti-angiogenic and anti-glioma therapy. Acta biomaterialia 2016, 38, 69-81, doi: 10.1016/j.actbio.2016.04.026.
Cai, S. X. Small molecule vascular disrupting agents: potential new drugs for cancer treatment. Recent Pat Anticancer Drug Discov 2007, 2, 79-101, doi: 10.2174/157489207779561462.
Chauhan, D. S.; Bukhari, A. B.; Ravichandran, G.; Gupta, R.; George, L.; Poojari, R.; Ingle, A.; Rengan, A. K.; Shanavas, A.; Srivastava, R.; et al. Enhanced EPR directed and Imaging guided Photothermal Therapy using Vitamin E Modified Toco-Photoxil. Sci Rep 2018, 8, 16673, doi: 10.1038/s41598-018-34898-3.
Cheng, Y. H.; He, C.; Riviere, J. E.; Monteiro-Riviere, N. A.; Lin, Z. Meta-Analysis of Nanoparticle Delivery to Tumors Using a Physiologically Based Pharmacokinetic Modeling and Simulation Approach. ACS nano 2020, 14, 3075-3095, doi: 10.1021/acsnano.9b08142.
Dubey, R. D.; Klippstein, R.; Wang, J. T.; Hodgins, N.; Mei, K. C.; Sosabowski, J.; Hider, R. C.; Abbate, V.; Gupta, P. N.; Al-Jamal, K. T. Novel Hyaluronic Acid Conjugates for Dual Nuclear Imaging and Therapy in CD44-Expressing Tumors in Mice In Vivo. Nanotheranostics 2017, 1, 59-79, doi: 10.7150/ntno.17896.
Fajardo, L. F.; Prionas, S. D. Endothelial cells and hyperthermia. Int J Hyperthermia 1994, 10, 347-353, doi: 10.3109/02656739409010278.
Garon, E. B.; Neidhart, J. D.; Gabrail, N. Y.; de Oliveira, M. R.; Balkissoon, J.; Kabbinavar, F. A randomized Phase II trial of the tumor vascular disrupting agent CA4P (fosbretabulin tromethamine) with carboplatin, paclitaxel, and bevacizumab in advanced nonsquamous non-small-cell lung cancer. Onco Targets Ther 2016, 9, 7275-7283, doi: 10.2147/OTT.S109186.
Grunberg J, Novak-Hofer I, Honer M, Zimmermann K, Knogler K, Blauenstein P, Ametamey S, Maecke H R, Schubiger P A. In vivo evaluation of 177Lu- and 67/64Cu-labeled recombinant fragments of antibody chCE7 for radioimmunotherapy and PET imaging of L1-CAM-positive tumors. Clin Cancer Res. 2005 Jul. 15; 11 (14): 5112-20.
Horsman, M. R. Tissue physiology and the response to heat. Int J Hyperthermia 2006, 22, 197-203, doi: 10.1080/02656730600689066.
Jin, J.; Xu, Z.; Zhang, Y.; Gu, Y. J.; Lam, M. H.; Wong, W. T. Upconversion nanoparticles conjugated with Gd (3+)-DOTA and RGD for targeted dual-modality imaging of brain tumor xenografts. Adv Healthc Mater 2013, 2, 1501-1512, doi: 10.1002/adhm.201300102.
Kim, J. H.; Kim, Y.; Bae, K. H.; Park, T. G.; Lee, J. H.; Park, K. Tumor-targeted delivery of paclitaxel using low density lipoprotein-mimetic solid lipid nanoparticles. Mol Pharm 2015, 12, 1230-1241, doi: 10.1021/mp500737y.
Kong, G.; Braun, R. D.; Dewhirst, M. W. Hyperthermia enables tumor-specific nanoparticle delivery: effect of particle size. Cancer Res 2000, 60, 4440-4445.
Madani, F.; Esnaashari, S. S.; Mujokoro, B.; Dorkoosh, F.; Khosravani, M.; Adabi, M. Investigation of Effective Parameters on Size of Paclitaxel Loaded PLGA Nanoparticles. Adv Pharm Bull 2018, 8, 77-84, doi: 10.15171/apb.2018.010.
Miller, M. A.; Chandra, R.; Cuccarese, M. F.; Pfirschke, C.; Engblom, C.; Stapleton, S.; Adhikary, U.; Kohler, R. H.; Mohan, J. F.; Pittet, M. J.; et al. Radiation therapy primes tumors for nanotherapeutic delivery via macrophage-mediated vascular bursts. Sci Transl Med 2017, 9, doi: 10.1126/scitranslmed.aal0225.
Mita, M. M.; Sargsyan, L.; Mita, A. C.; Spear, M. Vascular-disrupting agents in oncology. Expert Opin Investig Drugs 2013, 22, 317-328, doi: 10.1517/13543784.2013.759557.
Pedley, R B, J A Boden, R Boden, G M Boxer, A A Flynn, P A Keep, R H Begent Cancer Res. 1996 Jul. 15; 56 (14): 3293-300. Ablation of colorectal xenografts with combined radioimmunotherapy and tumor blood flow-modifying agents
Perrault, S. D.; Walkey, C.; Jennings, T.; Fischer, H. C.; Chan, W. C. Mediating tumor targeting efficiency of nanoparticles through design. Nano Lett 2009, 9, 1909-1915, doi: 10.1021/n1900031y.
Porcu, E.; Bortolozzi, R.; Basso, G.; Viola, G. Recent advances in vascular disrupting agents in cancer therapy. Future Med Chem 2014, 6, 1485-1498, doi: 10.4155/fmc. 14.104.
Rossin, R.; Versteegen, R. M.; Wu, J.; Khasanov, A.; Wessels, H. J.; Steenbergen, E. J.; Ten Hoeve, W.; Janssen, H. M.; van Onzen, A.; Hudson, P. J.; et al. Chemically triggered drug release from an antibody-drug conjugate leads to potent antitumour activity in mice. Nat Commun 2018, 9, 1484, doi: 10.1038/s41467-018-03880-y.
Shahin M, et al. Cancer Lett. 2013. PMID: 23073474. Engineered peptides for the development of actively tumor targeted liposomal carriers of doxorubicin.
Singh, V, Wang, S., Ng, K. W. Keratin as a biomaterial. In “Reference Module in Materials Science and Materials Engineering”, Chapter 2.25, p. 542-557, 2017. 10.1016/B978-0-12-803581-8.09317-6
Smolarczyk, R.; Czapla, J.; Jarosz-Biej, M.; Czerwinski, K.; Cichon, T. Vascular disrupting agents in cancer therapy. Eur J Pharmacol 2021, 891, 173692, doi: 10.1016/j.ejphar.2020.173692.
Song, C. W.; Kang, M. S.; Rhee, J. G.; Levitt, S. H. Effect of hyperthermia on vascular function in normal and neoplastic tissues. Ann N Y Acad Sci 1980, 335, 35-47, doi: 10.1111/j. 1749-6632.1980.tb50735.x.
Song, C. W.; Kim, M. S.; Cho, L. C.; Dusenbery, K.; Sperduto, P. W. Radiobiological basis of SBRT and SRS. Int J Clin Oncol 2014, 19, 570-578, doi: 10.1007/s10147-014-0717-z.
Zhong, J.; Wen, L.; Yang, S.; Xiang, L.; Chen, Q.; Xing, D. Imaging-guided high-efficient photoacoustic tumor therapy with targeting gold nanorods. Nanomedicine 2015, 11, 1499-1509, doi: 10.1016/j.nano.2015.04.002.
Zhou, Q.; Shao, S.; Wang, J.; Xu, C.; Xiang, J.; Piao, Y.; Zhou, Z.; Yu, Q.; Tang, J.; Liu, X.; et al. Enzyme-activatable polymer-drug conjugate augments tumour penetration and treatment efficacy. Nat Nanotechnol 2019, 14, 799-809, doi: 10.1038/s41565-019-0485-z.

1. A composition comprising nanoparticles comprising fibrinogen or a functional fragment thereof and a hydrophobic chemotherapeutic agent, wherein the average diameter of the nanoparticles is less than about 5 μm.

2. A composition comprising a hydrophobic chemotherapeutic agent, a protein, and a component having affinity for activated platelets that induces aggregates in serum at least 10 μm in size, wherein the average diameter of the nanoparticles is less than about 5 μm.

3. The composition of claim 2, wherein the component having affinity for activated platelets is fibrinogen or a functional fragment thereof.

4. The composition of any one of claims 1-3, wherein the fibrinogen and the chemotherapeutic agent are distributed throughout the nanoparticles.

5. The composition of any one of claims 1-4, wherein the fibrinogen and the hydrophobic chemotherapeutic agent are present on the surface of the nanoparticles.

6. The composition of any one of claims 1-5, wherein the fibrinogen and the hydrophobic chemotherapeutic agent are non-covalently associated in the nanoparticles.

7. The composition of any one of claims 1-6, wherein the ratio of fibrinogen to hydrophobic chemotherapeutic agent in the composition is about 20:1 to about 2:1.

8. The composition of any one of claims 1-7, wherein the ratio of fibrinogen to hydrophobic chemotherapeutic agent in the composition is to about 12:1 to about 4:1.

9. The composition of any one of claims 1-8, wherein the nanoparticles have a homogeneous structure.

10. The composition of any one of claims 1-9, wherein the nanoparticles further comprise a carrier protein.

11. The composition of claim 10, wherein the carrier protein is albumin or transferrin.

12. The composition of any one of claims 1-11, wherein the hydrophobic chemotherapeutic agent is selected from the group consisting of paclitaxel, camptothecin, docetaxel, and artemisinin.

13. The composition of any one of claims 1-12, wherein the nanoparticles further comprise an antibody that binds to a protein located on the surface of platelets.

14. The composition of any one of claims 1-13, wherein the average diameter of the nanoparticles in the composition is about 110 nm to about 400 nm.

15. The composition of any one of claims 1-14, wherein the composition does not comprise a denaturant.

16. The composition of any one of claims 1-15, wherein the nanoparticle further comprises a second chemotherapeutic agent.

17. The composition of any one of claims 1-16, further comprising a pharmaceutically acceptable excipient or buffer.

18. The composition of any one of claims 1-17, wherein the composition is a pharmaceutical composition.

19. The composition of any one of claims 1-18, wherein the composition is sterile.

20. A method of treating a solid tumor in an individual comprising administering i) a treatment causing vascular damage and ii) the composition of any one of claims 1-19 to the individual.

21. A method of creating embolism in tumors comprising administering the composition of any one of claims 1-19, wherein the nanoparticles are digested with the proteolytic enzymes released by necrotic cells, thereby releasing the drug.

22. The method of claim 20 or 21, wherein the coagulation cascade is activated.

23. The method of any one of claims 20-22, wherein the coagulation cascade is amplified.

24. The method of any one of claims 20-23, wherein a clot is formed in the tumor vasculature.

25. The method of any one of claims 20-24, wherein the nanoparticles bind to activated platelets.

26. The method of any one of claims 20-25, wherein each nanoparticle binds to two or more platelets.

27. The method of any one of claims 20-26, wherein the treatment causing vascular damage is selected from the group consisting of administering a vascular disrupting agent, applying radiation, X-rays, microwaves, infrared, radio frequencies, heat, ultrasound, mechanical insult, or antibody-drug conjugates that are targeted to the solid tumor.

28. The method of any one of claims 20-27, wherein the nanoparticles preferentially localize to the site of the solid tumor.

29. The method of any one of claims 20-28, wherein the nanoparticles form aggregates at the solid tumor site.

30. The method of any one of claims 20-29, wherein the aggregates are at least about 1 μm in size.

31. The method of any one of claims 20-30, wherein greater than about 10 mg/kg of fibrinogen is administered to the individual in the form of nanoparticles.

32. The method of any one of claims 20-31, wherein from about 10 mg/kg to about 500 mg/kg of fibrinogen is administered to the individual in the form of nanoparticles.

33. The method of any one of claims 20-32, wherein about 350 mg/kg of fibrinogen is administered to the individual.

34. The method of any one of claims 20-33, wherein the treatment causing vascular damage is selected from the group consisting of DMXAA, CA4P, Plinabulin, CKD-516, AVE8062, AVE9062 OXi4503, MPC6827, BNC105P, ABT-751, VEGF-gelonin, Verubulin, and flavone-8-acetic acid (FAA).

35. The method of any one of claims 20-34, wherein the hydrophobic chemotherapeutic agent is released at the site of the solid tumor.

36. The method of any one of claims 20-35, wherein the treatment causes cell lysis of cells within the tumor.

37. The method of any one of claims 20-36, wherein the treatment causes selective damage of endothelium associated with the solid tumor.

38. The method of any one of claims 20-37, wherein the hydrophobic chemotherapeutic agent is released from the nanoparticles by an active release process.

39. The method of any one of claims 20-37, wherein the hydrophobic chemotherapeutic agent is released from the nanoparticles by a passive release process.

40. The method of any one of claims 20-39, wherein an immune response is stimulated at the solid tumor.

41. The method of any one of claims 20-40, wherein the treatment does not cause prohibitive cell lysis or damage at normal tissue and/or wherein the hydrophobic chemotherapeutic agent is not prohibitively released at normal tissue.

42. The method of any one of claims 20-41, wherein the hydrophobic chemotherapeutic agent is administered at a higher level in the nanoparticle composition than the maximum tolerated dose for the hydrophobic chemotherapeutic agent administered in a non-nanoparticle formulation.

43. The method of any one of claims 20-42, wherein the hydrophobic chemotherapeutic agent is paclitaxel, and wherein 30 mg/kg to 90 mg/kg paclitaxel is administered to the individual in the form of nanoparticles.

44. The method of any one of claims 20-43, the vascular damaging agent or process is administered prior to, simultaneously with, or after the composition comprising nanoparticles.

45. The method of any one of claims 20-44, wherein the composition comprising nanoparticles is administered intravenously, intratumorally, or intraperitoneally.

46. The method of any one of claims 20-45, wherein the treatment causing vascular damage is administered intravenously, intratumorally, or intraperitoneally.

47. The method of any one of claims 20-46, wherein the solid tumor is selected from the group consisting of lung and bronchus, breast, prostate, colon, rectal, melanoma, bladder, kidney, endometrial, pancreatic, thyroid, liver, intrahepatic bile duct, gastrointestinal, brain and nervous system, cervical, head and neck, ovarian, testicular, eye, skin, lymphomas and bone and muscle sarcomas.

48. A kit comprising the composition of any one of claims 1-19 and a vascular disrupting agent.

49. A method of producing the composition of any one of claims 1-19, comprising sonicating the fibrinogen and the hydrophobic chemotherapeutic agent to produce nanoparticles.

50. The method of claim 49, further comprising, prior to the sonication, dissolving fibrinogen in a buffered solution and heating the dissolved fibrinogen at 37° C. for at least 5 minutes.
